SEQUENCE LISTING (26 sequences)

SEQ ID NO: 1 - DNA, 1686 bp, Artificial Sequence
  DNA sequence for Rluc-EYFP construct
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaacgggcccgggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 2 - DNA, 1708 bp, Artificial Sequence
  DNA sequence for Rluc-enterokinase-EYFP construct
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaacgggcccgggatccgggcgacgatgacgataagttggcggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaa

SEQ ID NO: 3 - DNA, 1710 bp, Artificial Sequence
  DNA sequence for Rluc-caspace-EYFP construct
atgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaacgggcccgggatccggccgacgaggtggacggcgaatccgcggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 4 - DNA, 1695 bp, Artificial Sequence
  DNA sequence for GFPRluc construct with linker
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggatcaagcttgcggtaccgcgggccctctagagccaccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccgctagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggcgcctgcctggccttccactacagctacgagcaccaggacaagatcaaggccatcgtgcacgccgagagcgtggtggacgtgatcgagagctgggacgagtggccagacatcgaggaggacatcgccctgatcaagagcgaggagggcgagaagatggtgctggagaacaacttcttcgtggagaccatgctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagatgttcatcgagagcgaccccggcttcttcagcaacgccatcgtggagggcgccaagaagttccccaacaccgagttcgtgaaggtgaagggcctgcacttcagccaggaggacgcccccgacgagatgggcaagtacatcaagagcttcgtggagagagtgctgaagaacgagcagtaa

SEQ ID NO: 5 - DNA, 1704 bp, Artificial Sequence
  DNA sequence for GFP1-caspase-3-Rluc construct
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggatcaagcttgggcgacgaggtggacggcgggccctctagagccaccatgaccagcaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccgctagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggcgcctgcctggccttccactacagctacgagcaccaggacaagatcaaggccatcgtgcacgccgagagcgtggtggacgtgatcgagagctgggacgagtggccagacatcgaggaggacatcgccctgatcaagagcgaggagggcgagaagatggtgctggagaacaacttcttcgtggagaccatgctgcccagcaagatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagatgttcatcgagagcgaccccggcttcttcagcaacgccatcgtggagggcgccaagaagttccccaacaccgagttcgtgaaggtgaagggcctgcacttcagccaggaggacgcccccgacgagatgggcaagtacatcaagagcttcgtggagagagtgctgaagaacgagcagtaa

SEQ ID NO: 6 - DNA, 6649 bp, Artificial Sequence
  DNA sequence for Rluc-PKA-EYFP construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaacgggcccgggatccgctgaggagggccagcctgggcgcggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaaagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - DNA, 1721 bp, Artificial Sequence
  DNA sequence for kemptide-GFP-Rluc construct
atgggcctgaggagggccagcctgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggatccagcttgcggtaccgcgggccctctagagccaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaa

SEQ ID NO: 8 - DNA, 1719 bp, Artificial Sequence
  DNA sequence for GFP-Rluc-kemptide construct
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggatccagcttgcggtaccgcgggccctctagagccaccatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaaagatctctgaggagggccagcctgggc

SEQ ID NO: 9 - DNA, 33 bp, Artificial Sequence
  sense strand of enterokinase site coding sequence
gatccgggcgacgatgacgataagttggcggta

SEQ ID NO: 10 - DNA, 26 bp, Artificial Sequence
  Antisense strand of enterokinase site coding sequence
cgccaacttatcgtcatcgtcgcccg

SEQ ID NO: 11 - DNA, 37 bp, Artificial Sequence
  sense strand of caspase-3 site coding sequence
gatccggccgacgaggtggacggcgaatccgcggtac

SEQ ID NO: 12 - DNA, 29 bp, Artificial Sequence
  Antisense strand of caspase-3 site coding sequence
cgcggattcgccgtccacctcgtcggccg

SEQ ID NO: 13 - DNA, 45 bp, Artificial Sequence
  linker sequence
tccggatcaagcttgcggtaccgcgggccctctagagccaccatg

SEQ ID NO: 14 - DNA, 29 bp, Artificial Sequence
  sense strand of caspase-3 site coding sequence
agcttgggcgacgaggtggacggcgggcc

SEQ ID NO: 15 - DNA, 21 bp, Artificial Sequence
  Antisense strand of caspase-3 site coding sequence
acccgctgctccacctgccgc

SEQ ID NO: 16 - protein, 7 aa, Artificial Sequence
  protein kinase A phosphorylation site
LRRASLG

SEQ ID NO: 17 - DNA, 34 bp, Artificial Sequence
  sense strand of Protein Kinase A site coding sequence
gatccgctgaggagggccagcctgggcgcggtac

SEQ ID NO: 18 - DNA, 26 bp, Artificial Sequence
  Antisense strand of Protein Kinase A site coding sequence
cgcgcccaggctggccctcctcagcg

SEQ ID NO: 19 - DNA, 29 bp, Artificial Sequence
  PCR Primer
catgccatgggccaccatggtgagcaagg

SEQ ID NO: 20 - DNA, 23 bp, Artificial Sequence
  PCR Primer
cgggatccggacttgtacagctc

SEQ ID NO: 21 - DNA, 56 bp, Artificial Sequence
  PCR Primer
cgggatccagcttgcggtaccgcgggccctctagagccaccatgacttcgaaagtt

SEQ ID NO: 22 - DNA, 30 bp, Artificial Sequence
  PCR Primer
gaagatctttgttcatttttgagaactcgc

SEQ ID NO: 23 - DNA, 39 bp, Artificial Sequence
  sense strand of kemptide coding sequence
catgggccaccatgggcctgaggagggccagcctgggcc

SEQ ID NO: 24 - DNA, 39 bp, Artificial Sequence
  Antisense strand of kemptide coding sequence
ccggtggtacccggactcctcccggtcggacccgggtac

SEQ ID NO: 25 - DNA, 27 bp, Artificial Sequence
  sense strand of kemptide coding sequence
gatctctgaggagggccagcctgggca

SEQ ID NO: 26 - DNA, 27 bp, Artificial Sequence
  Antisense strand of kemptide coding sequence
agactcctcccggtcggacccgtctag

(19) United States
(12) Patent Application Publication
(10) Pub. No.: US 2004/0214227 A1
(43) Pub. Date: Oct. 28, 2004

(54) Bioluminescence resonance energy transfer (bret) fusion molecule and method of use

(76) Inventor: Erik Joly, Blainville (CA)

Correspondence Address: Hale and Dorr, 60 State Street, Boston, MA, 02109, US

(21) Appl. No.: 10/168,443

(22) PCT Filed: Dec. 22, 2000
(86) PCT No.: PCT/CA00/01513

(30) Foreign Application Priority Data
Dec. 22, 1999 (CA) 2,292,036

(51) Int. Cl. G01N 33/53; C07K 14/00

(52) U.S. Cl. 435/7.1; 530/403

(57) ABSTRACT

This invention provides a bioluminescence resonance energy transfer (BRET) fusion molecule, and method of use. The fusion molecule comprises three components: a bioluminescent donor protein (BDP), a modulator, and a fluorescent acceptor molecule (FAM), wherein the FAM can accept energy from the BDP-generated luminescence when these components are in an appropriate spatial relationship and in the presence of an appropriate substrate. The modulator can either influence the proximity/orientation of the BDP and the FAM and thereby the energy transfer between these components, or it can play a different role in affecting the energy transfer between the BDP-generated activated product and the FAM.

### FIELD OF THE INVENTION

This invention pertains to the field of molecular interactions.

### BACKGROUND OF THE INVENTION

Interactions between proteins and other molecules play a key regulatory role in almost every biological process. Thus, many techniques have been developed to identify and characterize these interactions. These tools range from in vitro binding assays to library-based methods and include genetic methods such as searching for extragenic suppressors (Phizicky, E. M. & Fields, S., (1995) Microbiol. Rev., 59, 94-123).

One technique for assessing protein-protein interaction is based on fluorescence resonance energy transfer (FRET). In this process, one fluorophore (the “donor”) transfers its excited-state energy to another fluorophore (the “acceptor”) which usually emits fluorescence of a different color. According to Förster equation (Förster, T., (1948) Ann. Physik., 2, 55 and Förster, T., (1960) Rad. Res. Suppl., 2, 326), FRET efficiency depends on five parameters: (i) the overlap between the absorption spectrum of the second fluorophore and the emission spectrum of the first fluorophore, (ii) the relative orientation between the emission dipole of the donor and the absorption dipole of the acceptor, (iii) the distance between the fluorophores, (iv) the quantum yield of the donor and (v) the extinction coefficient of the acceptor.

FRET has been used to assay protein-protein proximity in vitro and in vivo by chemically attaching fluorophores such as fluorescein and rhodamine to pairs of purified proteins and measuring fluorescence spectra of protein mixtures or cells that were microinjected with the labeled proteins (Adams et al, (1991) Nature, 349, 694-697).

The cloning and expression of Green Fluorescent Protein (GFP) in heterologous systems opened the possibility of genetic attachment of fluorophores to proteins. In addition, the availability of GFP mutants with altered wavelengths (Heim et al., (1994) Proc. Natl. Acad. Sci. USA., 91, 12501-12504) allowed their use as FRET pairs.

An attractive application allowed by GFP-based FRET is the in vivo assay of protein interactions in organisms other than yeast. For example, fusion of GFP and BFP to the mammalian transcriptional factor Pit-1, showed homo-dimerization of Pit-1 in live HeLa cells (Periasamy, A. and Day, R. N., (1998) J. Biomed. Opt., 3, 1-7). In this type of assay, interactions can be examined in the proteins' native organism, such that cell-type specific modifications and/or compartmentalization of the proteins are preserved. Additionally, compartmentalization of these interacting proteins is potentially visible in the microscope.

FRET, however, has several limitations. As with any fluorescence technique, photobleaching of the fluorophore and autofluorescence of the cells/tissue can significantly restrict the usefulness of FRET, and in highly autofluorescent tissues, FRET is essentially unusable. Also, if the tissue is easily damaged by the excitation light, the technique may be unable to give a value for healthy cells. Finally, if the cells/tissues to be tested are photoresponsive (e.g., retina), FRET may be impractical because as soon as a measurement is taken, the photoresponse may be triggered.

### SUMMARY OF THE INVENTION

In one aspect, this invention provides a bioluminescence resonance energy transfer (BRET) fusion molecule that comprises three components: a bioluminescent donor protein (BDP), a modulator, and a fluorescent acceptor molecule (FAM), wherein the FAM can accept energy from the BDP-generated luminescence when these two components are in an appropriate spatial relationship and in the presence of the appropriate substrate. The modulator can either influence the proximity/orientation of the BDP and the FAM and thereby the energy transfer between these two components, or it can play a different role in affecting the energy transfer between the BDP-generated luminescence and the FAM. An interacting factor (IF) can either change the spatial relationship between the FAM and the BDP by interacting with (eg., bind to) the modulator directly (thereby causing either an increase or a decrease in the energy transfer between the BDP and the FAM) or it can interact with the modulator and in some manner enhance or quench the energy of the system; in either situation the interaction between the IF and the modulator ultimately causes in a change in the light emission (intensity and/or wavelength) from the system.

In another aspect, this invention provides a BRET system that comprises: 1) BRET fusion molecule, 2) an appropriate substrate to enable the BDP to generate an activated-product (i.e., the BDP-generated luminescence) that can transfer energy to the FAM, and 3) an IF that interacts with the modulator to cause a change in the light emission from the system.

In yet another aspect, this invention provides methods of using the BRET fusion molecule and the BRET system to assay for the availability (presence, absence, concentration, conformational state, bioavailability, etc) or activity of a target substance. The IF can be the target itself, or the IF can be sensitive to availability of an analyte in the system, such that the IF changes its interaction with the modulator in response to the availability of the analyte. Alternatively, determining the availability of the substrate can be the focus of the assay. The proximity of the components of the system might be dependent upon other factors in the biological assay, such as structural integrity of membrane walls, sequestration of factors by proteins, fluidity changes, pH changes, etc.

### BRIEF DESCRIPTION OF THE FIGURES

FIG. 1 is a pictorial description of a tandem embodiment of the BRET fusion molecule and system wherein the BDP and the FAM are attached to spacer elements that are attached to the modulator element.

FIG. 2 is a pictorial description of a non-tandem embodiment of the BRET fusion molecule and system, wherein the BDP and the FAM are attached to a spacer element that is attached to a modulator element.

FIG. 3 is a pictorial description of an embodiment of the BRET fusion molecule and system, wherein the BDP and the FAM are attached by an optional spacer element and the modulator is attached to: (3A) the BDP such that interaction with the IF causes a change in the conformational state of the BDP; or (3B) the FAM, such that interaction with the IF causes a change in the conformational state and activity of the FAM.

FIG. 4 is a pictorial description of an embodiment of the BRET fusion molecule and system, wherein the BDP and the FAM are attached are attached by an optional spacer element and the modulator is incorporated in: (4A) the BDP such that interaction with the IF causes a change in the activity of the BDP; or (4B) the FAM, such that interaction with the IF causes a change in the conformational state and activity of the FAM.

FIG. 5 shows the DNA sequence for the Rluc:EYFP construct (SEQ ID NO:1).

FIG. 6 shows the spectral analysis of the different Rluc/EYFP fusion configurations.

FIG. 7 shows the DNA sequence for the Rluc:enterokinase:EYFP construct (SEQ ID NO:2).

FIG. 8 shows the results of an in vitro enterokinase assay, demonstrating that only the ratio for the construct Rluc:enterokinase:EYFP decreases over time in the presence of the enterokinase enzyme (E).

FIG. 9 shows the DNA sequence for the Rluc:caspase:EYFP construct (SEQ ID NO:3).

FIG. 10 shows apoptosis induction using staurosporine in HeLa cells transfected with the apoptosis Rluc:Caspase:EYFP sensor.

FIG. 11 shows a DNA sequence (SEQ ID NO:4) encoding the GFP:Rluc fusion protein containing a unique 14 amino acid linker region between the GFP and the Rluc.

FIG. 12 shows the DNA sequence for GFP1:Caspase-3:Rluc construct (SEQ ID NO:5).

FIG. 13 demonstrates apoptosis induction using staurosporine in HeLa cells transfected with the GFP1:Caspase-3:Rluc sensor.

FIG. 14 is a pictorial description of a BRET assay using Rluc:PKA:EYFP for monitoring phosphorylation.

FIG. 15 shows the DNA sequence for the Rluc:PKA:EYFP construct (SEQ ID NO:6).

FIG. 16 shows the changes in the BRET ratio following the addition of forskolin to cells transfected with Rluc:PKA:EYFP.

FIG. 17 depicts the structure of the kemptide modulator (coding stand). The Kozak consensus is shown boldface type. The first methionine is shown with larger font and the nucleotide sequence coding for kemptide sequence is underlined.

FIG. 18 shows the DNA sequence for the kemptide:GFP:Rluc construct (SEQ ID NO:7).

FIG. 19 shows the DNA sequence for the GFP:Rluc:kemptide construct (SEQ ID NO:8).

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the laboratory procedures in spectroscopy, drug discovery, cell culture, molecular genetics, plastic manufacture, polymer chemistry, diagnostics, amino acid and nucleic acid chemistry, and sugar chemistry described below are those well known and commonly employed in the art. Standard techniques are typically used for preparation of plastics, signal detection, recombinant nucleic acid methods, polynucleotide synthesis, and microbial culture and transformation (e.g., electroporation, Calcium Chloride-heat shock).

The techniques and procedures are generally performed according to conventional methods in the art and various general references (see generally, Sambrook et al. Molecular Cloning: A Laboratory Manual, 2d ed. (1989) Cold Spring Harbor Laboratory Press, Cold Spring Harbor, N.Y., and Lakowicz, J. R. Principles of Fluorescence Spectroscopy, New York: Plenum Press (1983) for fluorescence techniques, which are incorporated herein by reference) which are provided throughout this document. Standard techniques are used for chemical syntheses, chemical analyses, and biological assays.

As employed throughout the disclosure, the following terms, unless otherwise indicated, shall be understood to have the following meanings:

“Bioluminescent donor protein (BDP)” refers to any protein capable of acting on a suitable substrate to generate luminescence. There are a number of different bioluminescent donor proteins that can be employed in this invention.

“Fluorescent acceptor molecule (FAM)” refers to any molecule which can accept energy emitted as a result of the activity of a bioluminescent donor protein, and re-emit it as light energy. There are a number of different fluorescent acceptor molecules that can be employed in this invention. The FAM may be proteinaceous or non-proteinaceous.

“Substrate” refers to any molecule that is employed by the bioluminescent donor protein to generate luminescence.

“Modulator” means a molecule or molecules that will undergo a change in response to an interaction with another molecule (called interacting factor), thereby affecting the proximity and/or orientation of the bioluminescent protein and the fluorophore. The term “sensor” is used to denote a modulator entity that performs a specific function, this term is used interchangeably with modulator, in a manner that denotes equivalent concepts.

“Interacting factor (IF)” refers a molecule either ions, second messenger, protein, protein domain, polypeptide or peptide capable of interacting with the modulator.

It is understood in the art that the BDP is an enzyme which converts a substrate into an activated product which then releases energy as it relaxes. Although the specification refers to the transfer of energy between the BDP and the FAM, it is understood that, technically, the activated product (generated by the activity of the BDP on the substrate) is the source of the BDP-generated luminescence that is transferred to the FAM. For the purpose of this invention, the orientation of the BDP relative to the FAM when it converts the substrate into the activated product is a crucial factor for the appropriate function of the invention.

“Protein” refers to include a whole protein, or fragment thereof, such as a protein domain or a binding site for a second messenger, co-factor, ion, etc. It can be a peptide or an amino acid sequence that functions as a signal for another protein in the system, such as a proteolytic cleavage site.

“Binding pair” refers to two moieties (e.g. chemical or biochemical) that have an affinity for one another. Examples of binding pairs include homo-dimers, hetero-dimers, antigen/antibodies, lectin/avidin, target polynucleotide/probe, oligonucleotide, antibody/anti-antibody, receptor/ligand, enzyme/ligand and the like. “One member of a binding pair” refers to one moiety of the pair, such as an antigen or ligand.

“Membrane-permeant derivative” refers a chemical derivative of a compound that has enhanced membrane permeability compared to an underivativized compound. Examples include ester, ether and carbamate derivatives. These derivatives are made better able to cross cell membranes, i.e. membrane permeant, because hydrophilic groups are masked to provide more hydrophobic derivatives. Also, masking groups are designed to be cleaved from a precursor (e.g., fluorogenic substrate precursor) within the cell to generate the derived substrate intracellularly. Because the substrate is more hydrophilic than the membrane permeant derivative it is now trapped within the cells.

“Isolated polynucleotide” refers a polynucleotide of genomic, cDNA, RNA or synthetic origin or some combination there of, which by virtue of its origin the “isolated polynucleotide” (1) is not associated with the cell in which the “isolated polynucleotide” is found in nature, or (2) is operably linked to a polynucleotide which it is not linked to in nature.

“Isolated protein” refers a protein of cDNA, recombinant RNA, or synthetic origin or some combination thereof, which by virtue of its origin the “isolated protein” (1) is not associated with proteins found it is normally found with in nature, or (2) is isolated from the cell in which it normally occurs or (3) is isolated free of other proteins from the same cellular source, e.g. free of human proteins, or (4) is expressed by a cell from a different species, or (5) does not occur in nature. “Isolated naturally occurring protein” refers to a protein which by virtue of its origin the “isolated naturally occurring protein” (1) is not associated with proteins that it is normally found with in nature, or (2) is isolated from the cell in which it normally occurs or (3) is isolated free of other proteins from the same cellular source, e.g. free of human proteins.

“Polypeptide” as used herein as a generic term to refer to native protein, fragments, or analogs of a polypeptide sequence. Hence, native protein, fragments, and analogs are species of the polypeptide genus.

“Naturally-occurring” as used herein, as applied to an object, refers to the fact that an object can be found in nature. For example, a polypeptide or polynucleotide sequence that is present in an organism (including viruses) that can be isolated from a source in nature and which has not been intentionally modified by man in the laboratory is naturally-occurring.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

“Control sequence” refers to polynucleotide sequences which are necessary to effect the expression of coding and non-coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, components whose presence can influence expression, and can also include additional components whose presence is advantageous, for example, leader sequences and fusion partner sequences.

“Polynucleotide” refers to a polymeric form of nucleotides of at least 10 bases in length, either ribonucleotides or deoxynucleotides or a modified form of either type of nucleotide. The term includes single and double stranded forms of DNA, RNA and combinations thereof.

“Polypeptide fragment” refers to a polypeptide that has an amino-terminal and/or carboxy-terminal deletion, but where the remaining amino acid sequence is usually identical to the corresponding positions in the naturally-occurring sequence deduced, for example, from a fall-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long.

“Plate” refers to a multi-well plate, unless otherwise modified in the context of its.

The term “test chemical” or “test compound” refers to a chemical to be tested by one or more screening method(s) of the invention as a putative candidate.

The terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), enzymatic labels (or reporter genes) (e.g., horseradish peroxidase, .beta.-galactosidase, .beta.-latamase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

“Analyte” refers to a substance for which the presence, absence, or quantity is to be determined. An analyte is typically a small molecule or ionic solute such as K⁺, H⁺, Ca²⁺, CO₂, Na⁺, Cl⁻, Mg², O₂, HCO₃, NO, and ATP.

Common examples of second messengers include cAMP, cGMP, Ca²⁺, IP₃, NO, DAG, ceramide and derivatives thereof, arachidonic acid and derivatives thereof and isoprenyl and derivatives thereof.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference).

This invention provides a bioluminescence resonance energy transfer (BRET) fusion molecule that comprises three components: a bioluminescent donor protein (BDP), a modulator, and a fluorescent acceptor molecule (FAM), wherein the FAM can accept energy from the BDP-generated luminescence when these components are in an appropriate spatial relationship and in the presence of the appropriate substrate. The modulator can either influence the proximity/orientation of the BDP and the FAM and thereby the energy transfer between the components, or it can play a different role in affecting the energy transfer between the BDP-generated luminescence and the FAM. An interacting factor (IF) can either change the spatial relationship between the FAM and the BDP by interacting with the modulator directly (thereby causing either an increase or a decrease in the energy transfer between the BDP-generated luminescence and the FAM) or it can interact with the modulator and in some manner enhance or quench the energy of the system; in either situation the interaction between the IF and the modulator ultimately causes in a change in the light emission (intensity and/or wavelength) from the system.

In another aspect, this invention provides a BRET system that comprises: 1) BRET fusion molecule, 2) an appropriate substrate to enable the BDP-generated luminescence, and 3) an IF that interacts with the modulator to cause a change in the light emission from the system.

In yet another aspect, this invention provides methods of using the BRET fusion molecule and the BRET system to assay for the availability (presence, absence, concentration, conformational state, bioavailability, etc) or activity of a target substance. The IF can be the target itself, or the IF can be sensitive to availability of an analyte in the system, such that the IF changes its interaction with the modulator in response to the availability of the analyte. Alternatively, determining the availability of the substrate can be the focus of the assay. The proximity of the components of the system may be dependent upon other factors in the biological assay, such as structural integrity of membrane walls, sequestration of factors by proteins, fluidity changes, pH changes, etc.

The BRET fusion molecule and system can be used in both in vivo and in vitro assays to detect molecular changes in a wide variety of applications, and is amenable to automation. In particular, it is useful for assaying protein interactions, enzyme activities and the concentration of analytes or signaling molecules in cells or in solution. It is useful in, for example, drug discovery, analyte screening, second messenger screening, drug screening, diagnosis, genotoxicity, identification of gene function, gene discovery, and proteomics.

The BRET Fusion molecule and System

Selecting Suitable Bioluminescent Donor Protein-Fluorescent Acceptor Molecule Pairs and Substrate

To design a system, it is first necessary to select a BDP and a FAM. In nature some bioluminescent protein-fluorophore pairings interact directly with one another. In other cases the pairing interaction is mediated through other proteins, in the absence of which interactive coupling occurs with a significantly lower affinity. If such a decreased affinity is present at expression levels required to achieve BRET, when the BDP and FAM are coupled respectively to other protein moieties, then such a pairing is suitable for BRET. Examples of such systems which occur naturally include the Renilla luciferase (Rluc)/Renilla GFP coupling (Cormier (1978) Methods Enzymol. 57:237-244) and the Aequorin/Aequorea GFP coupling (Wilson and Brand (1998) Annu. Rev. Cell Dev. Biol. 14:197-230).

One example of an engineered system which is suitable for BRET is an Rluc and enhanced yellow mutant of GFP (EYFP) pairing which do not directly interact to a significant degree with one another alone in the absence of mediating proteins (Y. Xu, et al. (1999) Proc. Natl. Acad. Sci. USA 96:151-156). Other fluorophores which are suitable pairings for Rluc include fluorophores from GFP classes 1, 2, 4 and some from 5. Specific examples of suitable fluorophores include but are not necessarily limited to: wild type GFP, Cycle 3, EGFP, Emerald, Topaz, 10C Q69K, and 10C.

There are a number of different bioluminescent donor proteins that can be employed in this invention. One very well known example is the class of proteins known as luciferases which catalyze an energy-yielding chemical reaction in which a specific biochemical substance, a luciferin (a naturally occurring fluorophore), is oxidized by an enzyme having a luciferase activity. A great diversity of organisms, both prokaryotic and eukaryotic, including species of bacteria, algae, fungi, insects, fish and other marine forms can emit light energy in this manner and each has specific luciferase activities and luciferins which are chemically distinct from those of other organisms. Luciferin/luciferase systems are very diverse in form, chemistry and function. For example, there are luciferase activities which facilitate continuous chemiluminescence, as exhibited by some bacteria and mushrooms, and those which are adapted to facilitate sporadic, or stimuli induced, emissions, as in the case of dinoflagellate algae. As a phenomenon which entails the transformation of chemical energy into light energy, bioluminescence is not restricted to living organisms, nor does it require the presence of living organisms. It is simply a type of chemiluminescent reaction that requires a luciferase activity which at one stage or another had its origins from a biological catalyst. Hence the preservation or construction of the essential activities and chemicals suffices to have the means to give rise to bioluminescent phenomena. Bioluminescent proteins with luciferase activity are thus available from a variety of sources or by a variety of means. Examples of bioluminescent proteins with luciferase activity may be found in U.S. Pat. Nos. 5,229,285, 5,219,737, 5,843,746, 5,196,524, 5,670,356.

Alternative BDPs that can be employed in this invention are enzymes which can act on suitable substrates to generate a luminescent signal. Specific examples of such enzymes are β-galactosidase, alkaline phosphatase, β-glucuronidase and β-glucosidase. Synthetic luminescent substrates for these enzymes are well known in the art and are commercially available from companies, such as Tropix Inc. (Bedford, Mass., USA).

It should be apparent to one skilled in the art that other non-GFP fluorophores, or other BDPs may be suitable for the BRET system of the present invention if the key pairing criteria are met. Other sources for example may include pairings isolated or engineered from insects (U.S. Pat. No. 5,670,356). Alternatively, it may be preferable for the bioluminescent protein with luciferase activity and the fluorophore to be derived from completely different sources in order to minimize the potential for direct affinity between the pairings.

There are a number of different FAMs that can be employed in this invention. One very well known example is the group of fluorophores that includes the green fluorescent protein from the jellyfish Aequorea victoria and numerous other variants (GFPs) arising from the application of molecular biology, eg. mutagenesis and chimeric protein technologies (R. Y. Tsien, (1998) Ann. Rev. Biochem. 63: 509-544). GFPs are classified based on the distinctive component of their chromophores, each class having distinct excitation and emission wavelengths: class 1, wild-type mixture of neutral phenol and anionic phenolate: class 2, phenolate anion: class 3, neutral phenol: class 4, phenolate anion with stacked π-electron system: class 5, indole: class 6, imidazole: and class 7, phenyl (R. Y. Tsien, (1998), supra). Further non-limiting examples of FAMs are provided in Table 1, which lists examples of non-protein FAMs paired with appropriate BDPs and substrates. Alternative FAMs are commercially available, for example, from companies such as Molecular Probes, Inc. (Eugene, Oreg. USA; as described in their Handbook of Fluorescent Probes and Research Chemicals: Haugland, E. P. ed.).

GFPs or other fluorophores, bioluminescent proteins with luciferase activities and the appropriate substrates have been combined into luminescence based assays, tagging, screening and protein-protein interaction detection systems: for example see U.S. Pat. Nos. 5,786,162, 4,614,712, 5,650,289, 5,837,465, 5,897,990, 5,702,883, 5,401,629, 5,221,623, 5,854,010, 5,770,391, 4,665,022, 5,854,004.

One criteria which should be considered in determining suitable pairings for BRET is the relative emission/fluorescence spectrum of the FAM compared to that of the BDP. The emission spectrum of the BDP should overlap with the absorbance spectrum of the FAM such that the light energy from the BDP luminescence emission is at a wavelength that is able to excite the FAM and thereby promote FAM fluorescence when the two molecules are in a proper proximity and orientation with respect to one another. For example it has been demonstrated that an Rluc/EGFP pairing is not as good as an Rluc/EYEF pairing based on observable emission spectral peaks (Y. Xu, (1999), supra and Wang, et al. (1997) in Bioluminescence and Chemiluminescence: Molecular Reporting with Photons, eds. Hastings et al. (Wiley, New York), pp. 419-422)

To study potential pairing, protein fusions are prepared containing the selected BDP and FAM and are tested, in the presence of an appropriate substrate. This may be achieved, for example, using the method of Xu et al., (1999) Proc. Natl. Acad. Sci. USA 96:151-6, or as described herein. Since the BDP and FAM are in close proximity to one another in the fusion molecule then they are a suitable pair if BRET is observed.

It should also be confirmed that the BDP and FAM do not spuriously associate with each other. This can be accomplished by separate co-expression of the BDP and FAM in the same cells and then monitoring the luminescence spectrum in order to determine if BRET occurs. This may be achieved, for example, using the method of Xu et al., (1999) Proc. Natl. Acad. Sci. USA 96:151-6, or as described herein. The selected BDP and FAM form a suitable BRET pair if little or no BRET is observed.

Further tests are conducted to determine whether the BDP and FAM will generate BRET when fused to interacting proteins. This can be performed by creating fusion molecules using the BDP and FAM with test proteins, known to interact with one another, and monitoring the luminescence spectrum after association. This may be achieved, for example, using the method of Xu et al., (1999) Proc. Natl. Acad. Sci. USA 96:151-6, or as described herein. The selected BDP and FAM form a suitable BRET pair if BRET is observed.

In one embodiment of the present invention Renilla luciferase (Rluc) (Cormier, (1978), Methods Enzymol., 57: 237-244), or one of its derivatives is used as the BDP with EYFP as the FAM. Rluc was selected because its emission spectrum is similar to that of cyan ECFP (λₘₐₓ=480 nm) which has been shown to exhibit FRET with EYFP (Miyawaki et al., (1997) Nature (London) 388: 882-887). The coelenterazine substrate for Rluc is a hydrophobic molecule that is permeable to most cell types.

The Modulator

A modulator is a peptide, protein, nucleic acid or other synthetic or natural molecule that can interact with a separate entity or type of entity, the IF, and thereby initiate a sequence of events that ultimately results in a change in light emission from the BRET system or fusion molecule of the present invention. The modulator can either influence the proximity/orientation of the BDP and the FAM and thereby the energy transfer between these two components, or it can otherwise affect the energy transfer between the BDP and the FAM.

When the modulator is designed to influence the spatial relationship between the FAM and the BDP, interaction of the IF with the modulator causes a change in the proximity and/or orientation between the BDP and the FAM, thereby causing either an increase or a decrease in the energy transfer between the BDP-generated luminescence and the FAM, ultimately resulting in a change in the energy emission from the system.

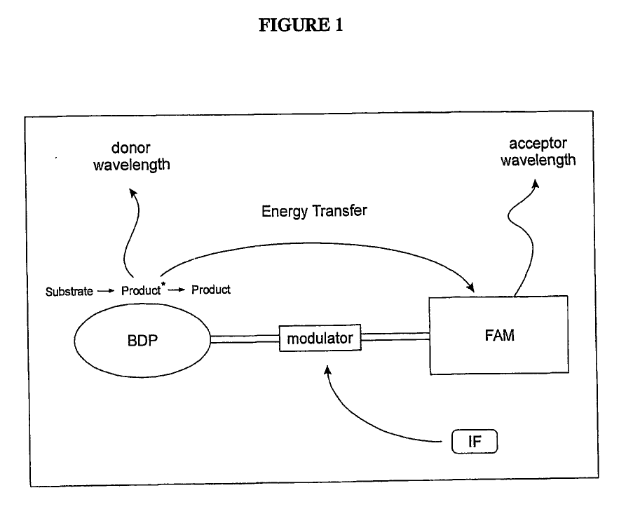

In one embodiment, using the BRET fusion molecule depicted in FIG. 1 for example, interaction of an IF with the modulator can result in cleavage of the modulator, for example when the modulator is a recognition/cleavage site for a protease, and the appropriate protease is the IF of interest. Alternatively, a cofactor, substrate or condition such as pH or the media, that is necessary for the activity of the protease, could be the target of the assay, such that when the target (cofactor, substrate, or optimal pH) is present, the protease is activated to cleave the modulator, resulting in a change in the energy emission from the system.

In another example, a modulator composed of specific proline-rich sequence is recognized by proteins having a SH3 domain. Upon binding of the protein onto the modulator, a conformation change in the overall structure of the fusion molecule occurs. This conformation change affect the spatial relationship (distance and orientation) of the BDP and the FAM and consequently alters the energy emission from the system.

In another embodiment, using the BRET fusion molecule depicted in FIGS. 1, or 2 for example, interaction of an IF with the modulator can lead to chemical modification of the modulator, for example phosphorylation, which would result in a conformational change in the modulator. This conformational change would translate into a change in the orientation and/or proximity between the BDP and the FAM, causing a change in the efficiency of the energy transfer between the BDP-generated luminescence and the FAM, resulting in the energy emission from the system.

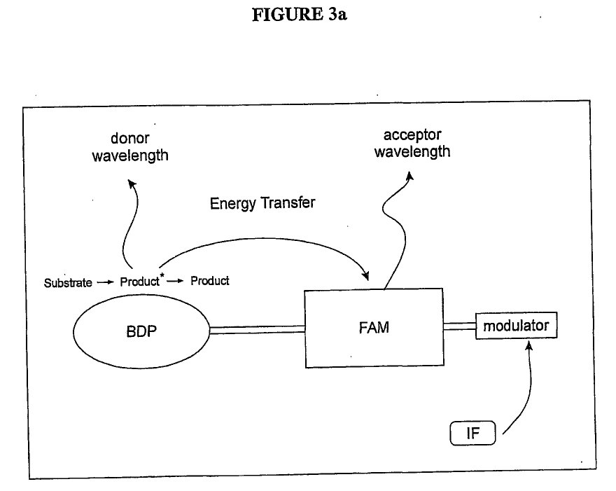

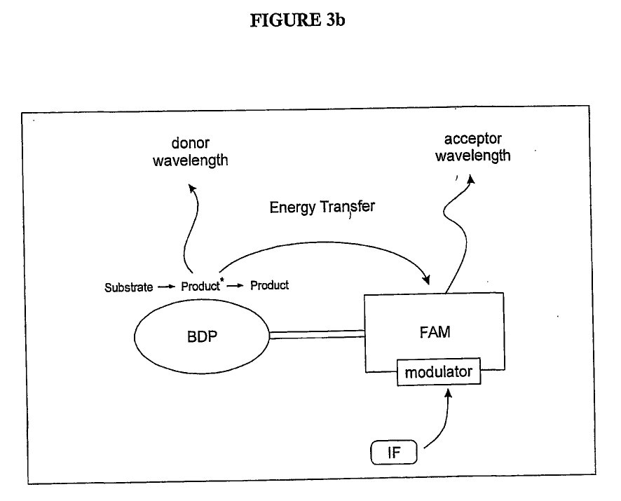

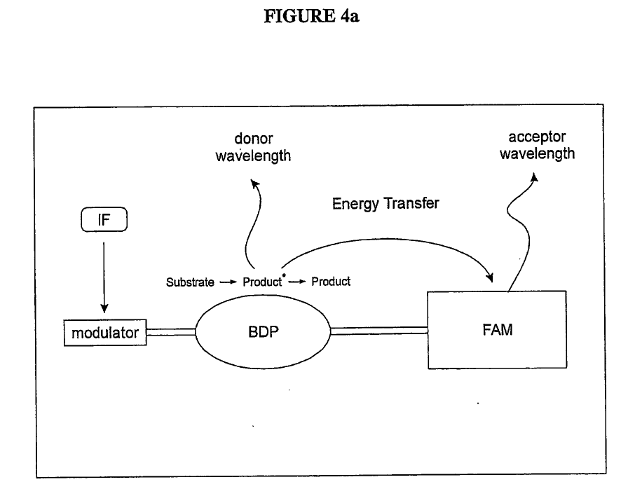

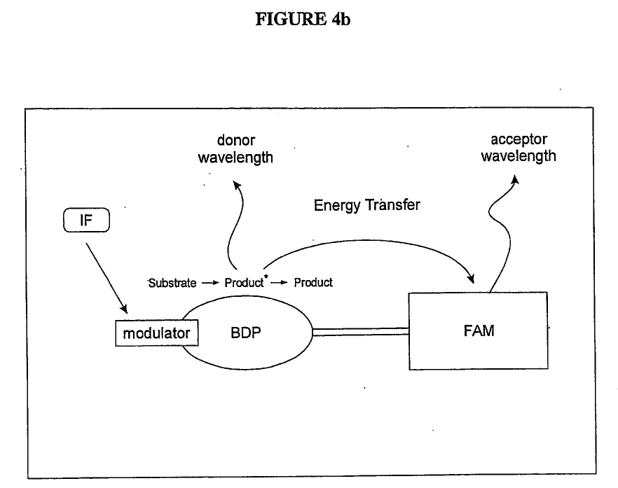

In another embodiment, using the BRET fusion molecule depicted in FIGS. 3 or 4 for example, interaction of an IF with the modulator can lead to chemical modification of the modulator, for example by phosphorylation, which would result in a conformational change in either the BDP or the FAM, causing a change in the efficiency of the energy emission from the system.

Examples of modulators with means to affect spatial relationship include, a protease recognition/cleavage site, a second messenger binding site, an ion binding molecule, a homo-dimer or hetero-dimer subunit, a nucleic acid sequence, a kinase substrate or other enzyme substrate, a receptor, a receptor fragment or subunit, or a receptor ligand.

When the modulator is designed to play role that is different from primarily affecting the spatial relationship, it can be designed to interact with the IF in the manner of a binding site for the IF. In one example, the IF is a molecule with enhancer means or quenching means, such that upon binding to the modulator, the efficiency of energy transfer in the system is effected, increased or decreased, respectively.

In another embodiment the modulator can be a single entity or two entities (covalently attached to each other and to both the BDP and the FAM) that interact with various types IFs.

The Interacting Factor (IF)

In one embodiment of the present invention, the modulator is recognized by an IF that acts as a quencher molecule(s). Binding of IF quencher molecules to the modulator, either directly or indirectly, interferes with the energy transfer between the BDP and the FAM. Interaction of the IF quencher with the modulator does not always change the distance between the BDP and the FAM or alter their relative orientation. Instead the IF quencher can directly absorb energy without any re-emission of light and thereby decrease energy transfer efficiency between the BDP and the FAM. In the case of direct binding, the quencher and the IF are the same entity. In the case of indirect interaction, the quencher is covalently attached to the IF.

In a similar embodiment of the present invention the IF acts as a blocking fluorophore, wherein it includes any molecule that can interact directly or indirectly with the modulator and upon interaction can absorb energy from the BDP-generated luminescence. A blocking fluorophore differs from a quencher since it re-emits light at a different wavelength than the FAM. The blocking fluorophore re-emission can be specifically measured using filter adapted for the blocking fluorophore emission wavelength. Contrary to the FAM, the blocking fluorophore is not covalently attached to the bioluminescent protein. In the case of direct binding, the blocking fluorophore and the IF are the same entity. In the case of indirect interaction, the blocking fluorophore is covalently attached to the IF.

In another embodiment the IF can act as an enhancer, wherein it includes any molecule that can interact directly or indirectly with the modulator and upon interaction can increase energy transfer from the BDP to the FAM. An example of an enhancer is a highly charged molecule than could change, upon binding to the modulator, the overall polarity of the BDP-modulator-FAM fusion molecule. In the case of direct binding, the enhancer and the IF are the same entity. In the case of indirect interaction, the enhancer is covalently attached to the IF.

In another embodiment, the IF is an enzyme that will recognize and modify the modulator in the fusion molecule of the present invention. For example the IF can be kinase that will recognize and phosphorylate the modulator. Kinases are proteins that recognize a specific amino acid sequence (like the protein kinase A recognition site also known as kemptide sequence: LRRASLG in single amino acid code) and attach a phosphate group to a specific residue. Addition of polar group like a phosphate, can result in a change in either the distance between the BDP and the FAM, through electrostatic attraction/repulsion or steric congestion between these moieties, or in a change of the relative orientation of these two moieties. Addition of phosphate group can also perturb the energy transfer efficiency through a change of the overall polarity or hydrophobicity of the fusion molecule.

In a similar embodiment, the modulator can be recognized and modified by any type of transferase enzymes. These enzymes are known to transfer specific groups, such as, but not limited to, sugars, lipids, methyl, ubiquitin or any derivative thereof, to an amino acid within a specific recognition sequence. Addition of these groups to the modulator, can result in a change in either the distance between the BDP and the FAM, through electrostatic attraction/repulsion or steric congestion between these moieties, or in a change of the relative orientation of these two moieties. Addition of these group could also perturb the energy transfer efficiency through a change of the overall polarity or hydrophobicity of the fusion molecule of the present invention.

In an alternative embodiment, the modulator can be recognized and be cleaved by proteases. Proteases are known to recognize and cleave specific amino acid sequences. If these sequences are introduced either genetically or synthetically in the modulator region, they can be recognized and be cleaved by the action of these proteases. Upon cleavage of the modulator, the BDP and the FAM are separated, energy transfer does not occur and there is a resulting decrease in the FAM light output.

Arrangement of the BRET Fusion Molecule Components

One embodiment of the BRET fusion molecule is exemplified in FIG. 1, which presents a pictorial description of a tandem embodiment of the BRET fusion molecule and system wherein the BDP and the FAM are attached to spacer elements that are attached to the modulator element.

One typical example of this embodiment is the use of the fusion molecule of the present invention as a sensor that will detect changes in a component of an environment. For example, a change in the environment can cause the sensor to undergo a conformational change that affects the spatial relationship of the BDP and the FAM, that is signaled by the light emission from the system.

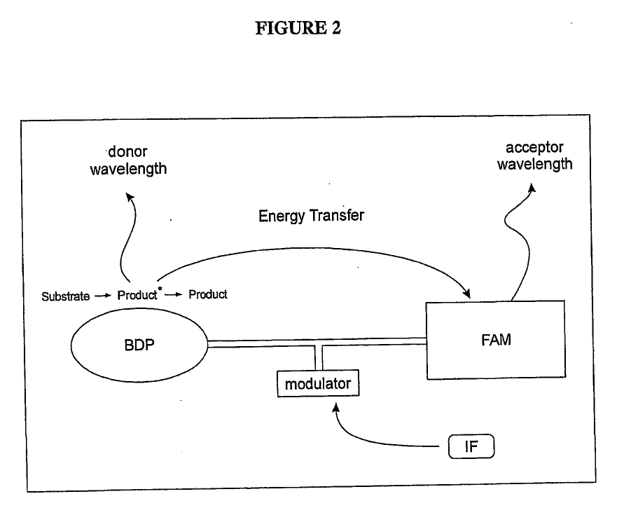

Another embodiment of the BRET fusion molecule is exemplified in FIG. 2 which presents a pictorial description of a non-tandem embodiment of the BRET fusion molecule and system, wherein the BDP and the FAM are attached to a spacer element that is attached to a modulator element.

Another embodiment of the BRET fusion molecule is exemplified in FIG. 3 which presents a pictorial description of an embodiment of the BRET fusion molecule and system, wherein the BDP and the FAM are attached by an optional spacer element and the modulator is attached to either the BDP (FIG. 3A) such that interaction with the IF causes a change in the conformational state and/or activity of the BDP; or the FAM (FIG. 3B), such that interaction with the IF causes a change in the conformational state and/or activity of the FAM.

Another embodiment of the BRET fusion molecule is exemplified in FIG. 4 which presents a pictorial description of an embodiment of the BRET fusion molecule and system, wherein the BDP and the FAM are attached by an optional spacer element and the modulator is incorporated in either the BDP (FIG. 4A) such that interaction with the IF causes a change in the conformational state and/or activity of the BDP; or the FAM (FIG. 4B), such that interaction with the IF causes a change in the conformational state and/or activity of the FAM.

The Use of the BRET System for Cell-Based Assays

The BRET system of the present invention is useful for cell-based assays. In general, the fusion molecules are introduced by transforming or transfecting a cell with one or more vectors comprising the recombinant DNA encoding some, or all, of the components of the system. The cell expresses the fusion molecule and BRET will occur when the BDP, the FAM and the substrate are in the appropriate spatial relationship. The substrate can be either added or, if it is protein or nucleic acid based, introduced in its nucleic acid form, such as via a vector with an inducible promoter.

The components of the BRET system and their derivatives, can be expressed either transiently or stably in various cell types (mammalian, yeast, bacteria, insect or using any virus expression system) using recombinant techniques known in the field. If the IF endogenous in the cell type then it is necessary to add the fusion molecule, either directly or via transfection with the gene encoding the fusion molecule. If not, the fusion molecule and the IF are co-transfected/co-transformed/co-expressed. Cells are maintained for the duration of the assay. The assay can be performed at various temperatures and culture conditions. At the time of the assay, the bioluminescent reaction can be initiated by adding the substrate to the cell mixture before light emission is detected and measured.

The Use of the BRET System for in vitro Assays

The BRET system can be used for cell-free in vitro assays, wherein the components can be made, purified and then combined in an in vitro assay, such as in a microtiter well. One or more of the components can be attached to a solid support such as a microtiter well, polyester cloth or polymacron cloth.

The Use of the BRET System for Chemical Detection

There are several applications that make use of the FRET ratio principle, e.g., for measuring gene expression (Zlokarnik et al., 1998) or for measuring intracellular levels of calcium ions (Romoser et al., 1997; Miyawaki et al., 1997). BRET could be used in these applications, and may in fact have advantages over using FRET. For example, two groups have reported a novel method for ratio imaging of Ca²⁺ based on using two GFP variants (e.g., BFP, CFP, GFP, YFP) linked-by a Ca²⁺-binding calmodulin sequence (Romoser et al., 1997; Miyawaki et al., 1997). When the linker sequence binds Ca²⁺; it undergoes a conformational change that brings the GFP variants closer together, enabling FRET. In the absence of sufficient Ca²⁺, the GFP variants remain far apart and FRET does not occur. Therefore, the magnitude of FRET is an indicator of Ca²⁺ levels; because this method is based on a ratio, differences in the level of expression of the Ca²⁺ indicator do not introduce artifacts to the measurement.

This principle is adaptable to BRET by designing a construct where a BDP, such as Rluc, is linked to a FAM, such as EYFP, via the same calmodulin linker. Using Rluc and EYFP, the ratio of luminescence at 530 nm versus 480 nm should give an estimate of Ca²⁺. The BRET system should be better than a FRET system for measuring Ca²⁺ in photoresponsive or auto-fluorescent tissue, and there will be no photobleaching of fluorophores. Furthermore, because there will be no direct excitation of the acceptor fluorophore, an improved ratio measurement of Ca²⁺ can be obtained with the BRET system of the present invention.

The Use of the BRET Fusion System with Automation and High-Throughput Screening

The BRET system is adaptable to means of automation and high-throughput screening, using appropriate instrumentation. Using an imaging instrument similar to the described in Xu, et al. (1999) Proc. Natl. Acad. Sci., 96, 151-156, it is possible to screen colonies of bacteria or yeast on agar plates. Alternatively, a photomultiplier-based instrument designed to measure luminescence of liquid cultures in 96-well plates can be adapted for high-throughput BRET screening by insertion of switchable interference filters that correlate with the emission wavelengths for any BRET pair, in front of the photomultiplier tube.

It is also contemplated that each of these embodiments can be partly or wholly incorporated into a kit format for distribution. The BRET system can be presented in a commercially packaged form; a packaged combination of one or more containers, devices, or the like, holding the necessary reagents and usually including written instructions describing the performance of the assay procedure. Reagent systems of the present invention involve all possible configurations and compositions for performing the various BRET assays described herein.

One skilled in the art will readily recognize that the system of the present invention can readily be incorporated into one of the established kit formats which are well known in the art.

Preparing the BRET Fusion Molecule and System

The basic components of the BRET system (BDP, FAM and modulator) and their derivatives, can be produced either using standard recombinant DNA technology, chemically synthesis, isolation from natural sources or any combination thereof. Regardless of the method of preparation, the following configurations of the BRET fusion molecule are contemplated.

BDP-M-FAM (FIG. 1): the modulator is genetically or chemically inserted in between the BDP and FAM moieties;

FAM-M-BDP: inverse configuration of the BDP-M-FAM;

BDP-_{M}-FAM (FIG. 2): T variation, in which the modulator is chemically attached to a linker (peptide or non-peptide) molecule attaching the BDP and FAM moieties together;

FAM-_{M}-BDP: inverse configuration of the BDP-M-FAM;

BDP-FAM-M (FIG. 3a): the modulator is genetically or chemically attached to the FAM moiety only;

BDP-FAM(M) (FIG. 3b): the modulator is genetically inserted either at the amino- or the carboxy- of the FAM moiety or internally in the FAM moiety;

M-BDP-FAM (FIG. 4a): the modulator is genetically or chemically attached to the BDP moiety only;

BDP(M)-FAM (FIG. 4b): the modulator is genetically inserted either at the amino- or the carboxy-terminus of the BDP moiety or internally in the BDP moiety.

In Vivo Preparation of the BRET Fusion Molecule

In one embodiment of the present invention, the BRET fusion molecule is produced entirely by recombinant DNA technology. The BDP and the FAM are selected, as described herein, such that, with the addition of the appropriate substrate, the fusion molecule product can exhibit BRET. When using recombinant DNA technology to produce the fusion molecule the FAM must be a fluorescent protein.

As described herein, the modulator comprises the sequence required for the biological/biochemical activity alone (e.g. calmodulin sequence that binds Ca²⁺) and it can additionally comprise spacer sequences on one or both sides of the sequence required for biological/biochemical activity. Spacer sequences can facilitate efficient recognition of the biological/biochemical activity sequence by the IF.

The fusion molecule of the present invention can be engineered to comprise additional amino acid sequences at the amino- and/or the carboxy-terminus, internally (in the modulator, BDP or FAM) or any combination thereof, of the BRET fusion molecules, that can be used, for example, to target the fusion molecule, facilitate purification of the fusion molecule, facilitate immobilization of the fusion molecule, improve stability of the fusion molecule, etc. For example peptide leader sequences from known proteins can be added to target the fusion molecule to various locations in a cell and His-tag sequences or protein A sequences can be added to aid in purification or to facilitate immobilization.

Various fusion DNA constructs that can express the fusion molecule of the present invention, as depicted in FIGS. 1, 3b and 4b), can be produced using recombinant DNA technology. First the coding sequences for the various components listed above are determined. These sequences can be obtained from publicly available databases, such as Genbank, or can be determined from isolated proteins and peptides using standard techniques known in the art. The coding sequences are then introduced into an expression vector. Commercially available expression vectors have been engineered with multiple cloning sites that contain a series of restriction enzyme recognition/cleavage sites that are used to introduce heterologous DNA in frame with regulatory sequences. The coding sequences may require modification, for example by PCR, to incorporate appropriate restriction enzyme recognition/cleavage sites at the 3′ and 5′ ends.

Most commercially available expression vectors have all the necessary control sequences to efficiently control transcription and therefore protein expression of the fusion DNA construct inserted. Other commercially available expression vectors are designed to allow cloning of alternative regulatory sequences, upstream and downstream of the inserted DNA construct. Expression vectors can be either purchased from a commercial supplier (e.g. Promega, Invitrogen, Stratagen) or engineered in house using DNA recombinant technology. Expression vectors can be adapted for use in prokaryotic or eukaryotic cells using appropriate control sequences and sequence elements required for the replication and selection of the expression vector and expression of the protein.

Expression vectors harboring the fusion DNA sequence can be transfected or transformed into eukaryotic or prokaryotic cell, respectively, for expression of the fusion DNA. Transfection in eukaryotic cells involves the use of standard techniques known in the art, such as, but not limited to, electroporation, calcium phosphate, lipid-based transfection, micro-injection, polymer-based transfection. The various configurations of the fusion molecule can be also expressed in prokaryotic cells using standard technique for the introduction of DNA in those cell types, such as, but not limited to, electroporation, calcium chloride-heat shock technique. Successful transfection or transformation can be determined using reporter genes incorporated in the expression vector (e.g. β-lactamase gene, β-galactosidase gene, neomycin resistance, etc.).

Finally, with successful transfection or transformation of the recombinant expression vector the cells can express the BRET fusion molecule (as depicted in FIGS. 1, 3b and 4b). Thus, in one embodiment of the present invention, the fusion molecule is produced in vivo and subsequently used in vivo for a BRET assay, if cells express or contain (endogenously or exogenously) the IF. The substrate (e.g. coelenterazine when the BDP is a luciferase or aequorin) is used to initiate the bioluminescent reaction (BRET assay). Various substrates, such as coelenterazine, can be purchased from commercial suppliers (e.g. Molecular Probes, Biosynth, Proleume) or synthesized.

The construction of a fusion DNA sequence and an expression vector, and the expression of the fusion molecule product involve the use of recombinant techniques well known in the art (see, for example, Sambrook, J., Fritsch, E. F. & Maniatis, T.; Molecular Cloning: A laboratory manual, 2^{nd} ed. (1989) Cold Spring Harbor Laboratory Press; and Ausubel, F. M. et al.; Current Protocols in Molecular Biology, Vol. 1 (1995) John Wiley & Sons, Inc).

In another embodiment of the present invention, the BRET fusion molecule (depicted in FIGS. 1, 3b and 4b) is produced in vivo as one entity, as described above, and subsequently isolated and used in an in vitro BRET assay. The DNA construct for a selected configuration of the BRET fusion molecule, is transfected, or transformed, into suitable cell lines (eukaryotic or prokaryotic) for its production only. The BRET fusion molecules are expressed in the cells and are then purified or semi-purified from the transfected or transformed cells. One exemplary method of purification of a BRET fusion molecule is described in Example I, in which a BDP-modulator-FAM molecule is purified from a cell extract using affinity chromatography. In this example a His-tag coding sequence was engineered in the DNA construct (such that the poly-histidine sequence was at the amino-terminus) that facilitated the purification of the resulting fusion molecule by making use of the affinity of the tandem histidines for Nickel on a chromatography support. Standard biochemical techniques can be also used alone or in combination with affinity chromatography to purify fusion molecules of the present invention to the desired purity.

It would be apparent to one skilled in the art that the modulator of the purified or semi-purified fusion molecules can be further chemically or enzymatically modified before their use in a BRET assay. For example, a phosphate group can be added to a specific amino acid residue found in the modulator sequence (e.g. tyrosine), thereby allowing a given IF to recognize the modulator. The phosphate group can be added enzymatically, using a tyrosine or serine/threonine kinase, or chemically, using, for example, a phosphoramidite reagent. Other non-limiting examples of modifications are methylation, isoprenylation, addition of palmitate, myristate, sulfate, sugar groups).

In an alternative embodiment a BRET fusion molecule having the configuration depicted in FIG. 2, wherein the modulator is chemically attached to a linker molecule (peptide in this case) attaching the BDP and FAM moieties together, is produced using a combination of in vivo and in vitro methods. First a fusion molecule having a peptide linker between the BDP and FAM (BDP-FAM or FAM-BDP) is genetically engineered and expressed in cells using standard recombinant techniques. The fusion molecule is then purified or semi-purified before chemically or enzymatically attaching the modulator into the linker region. It should be apparent to one skilled in the art that this modulator can be peptide-based or chemically-based. This configuration can be used in an in vitro assay or an in vivo assay, using various techniques such as protein transfection techniques, permeabilizing agent, by micro-injection or electroporation to introduce the protein inside the cells. In each cases, the substrate is used to initiate the bioluminescent reaction (BRET assay). The substrate can be purchase from commercial suppliers (e.g. Molecular Probes), or synthesized.

The modulator can be inserted within the coding region of either BDP or FAM. This can be achieved, for example, by ligating a coding sequence directly within or adjacent to the BDP or FAM coding sequence or by using site-specific mutagenesis to introduce a modulator sequence. Many site-specific mutagenesis kits are now commercially available (e.g. Quickchange from Stratagen). The choice of the site of insertion within the BDP or FAM sequence can be random or site specific based on the structure of the BDP or the FAM.

In Vitro Preparation of the BRET Fusion Molecule

In one embodiment of the present invention the BRET fusion molecule (as depicted in FIGS. 1, 3b and 4b) can be synthesized as one entity in vitro using standard in vitro transcription-translation techniques (IVTT) known in the art. When produced in vitro, a DNA fusion construct is prepared, as indicated above, using standard techniques that encodes a BRET fusion molecule (as depicted in FIGS. 1, 3b and 4b). The DNA fusion construct is engineered with an appropriate upstream promoter (e.g. a viral-based promoter such as T7, T3, SP6 which is recognized by specific viral RNA polymerases) and other regulatory sequences. The DNA fusion construct may be linear or circular. The DNA fusion construct is then used to produce mRNA in vitro, which are subsequently used in an in vitro translation reaction using cell extracts (e.g. E. coli, wheat germ or rabbit reticulocytes) to produce the BRET fusion molecule. The in vitro transcription and translation may be performed in a single reaction mixture (coupled) or in separate reaction mixtures. These techniques of producing large quantities of specific mRNA and proteins in vitro are well known in the art. Furthermore, several suppliers (e.g. Promega, Invitrogen, Life-Technologies, Stratagen) provide kits and reagents necessary to perform these experiments.

If required, the BRET fusion molecules produced using IVTT can be purified or semi-purified before use in a BRET assay. One exemplary method of purification of a BRET fusion molecule is described in Example I, in which a BDP-modulator-FAM molecule is purified from a cell extract using affinity chromatography. In this example a His-tag coding sequence was engineered in the DNA construct (such that the poly-histidine was at the amino-terminus) that facilitated the purification of the resulting fusion molecule by making use of the affinity of the tandem histidines for Nickel on a chromatography support. Standard biochemical techniques can be also used alone or in combination with affinity chromatography to purify fusion molecules of the present invention to the desired purity.

It would be apparent to one skilled in the art that the modulator of the purified, semi-purified or unpurified fusion molecules can be further chemically or enzymatically modified before their use in a BRET assay. For example, a phosphate group can be added to a specific amino acid residue found in the modulator sequence (e.g. tyrosine), thereby allowing a given IF to recognize the modulator. The phosphate group can be added enzymatically, using a tyrosine or serine/threonine kinase, or chemically, using, for example, a phosphoramidite reagent. Other non-limiting examples of modifications are methylation, isoprenylation, addition of palmitate, myristate, sulfate, sugar groups).

Finally, the purified, semi-purified unpurified BRET fusion molecule (modified or unmodified) once produced in vitro, can be used in an in vitro assay or in an in vivo assay, using various techniques such as protein transfection techniques, permeabilizing agent, by micro-injection or electroporation to introduce the protein inside the cells. In all cases, the substrate is used to initiate the bioluminescent reaction (BRET assay). The substrate can be purchased from commercial suppliers (e.g. Molecular Probes) or synthesized.

In an alternative embodiment a BRET fusion molecule having the configuration depicted in FIG. 2, wherein the modulator is chemically attached to a linker molecule (peptide in this case) attaching the BDP and FAM moieties together, is produced using a combination of in vitro methods. First a fusion molecule having a peptide linker between the BDP and FAM (BDP-FAM or FAM-BDP) is genetically engineered and expressed IVTT techniques. The fusion molecule is then purified or semi-purified before chemically or enzymatically attaching the modulator into the linker region. It should be apparent to one skilled in the art that this modulator can be peptide-based or chemically-based. This configuration can be used in an in vitro assay or an in vivo assay, using various techniques such as protein transfection techniques, permeabilizing agent, by micro-injection or electroporation to introduce the protein inside the cells. In each cases, the substrate is used to initiate the bioluminescent reaction (BRET assay). The substrate can be purchase from commercial suppliers (e.g. Molecular Probes), or synthesized.

Preparation of the BRET Fusion Molecule from Coupling of Separate BDP and FAM Entities

In one embodiment of the present invention, the BDP and the FAM moieties of the BRET fusion molecule are produced separately (two different entities) and attached together before performing the BRET assay. A worker skilled in the art would appreciate that it is not necessary for the two entities to be prepared using the same techniques prior to their attachment to one another.

In one embodiment one or both of the BDP and FAM are produced and purified using recombinant technology as described previously. Briefly, genes encoding the BDP and/or the FAM are cloned into separate expression vectors and are then used to transform or transfect cells which will produced the proteins. E. coli, yeast and insect cells are useful to produce high amount of a protein of interest (i.e. the BDP and FAM). Several kits and reagent are commercially available to express and produce a given protein using a specific cell type. The BDP and the FAM are purified or semi-purified before attachment to one another. Methods of purification of GFPs, which can be used as FAMs, and luciferases, which can be used as BDPs, are known in the art (Deschamps, et al, (1995) Protein Expression and purification 6: 555-558; Matthews, J. C., et al (1977) Biochemistry 16: 85-91; Ward, W. W. and Cormier, M. J. (1978) Photochemistry and Photobiology 27: 389-396).

In an alternative embodiment one or both of the BDP and the FAM are produced using IVTT as described above for the fusion molecule.

In another alternative embodiment one or both of the BDP and the FAM can be isolated from natural sources, and purified or semi-purified before chemical attachment to one another. As indicated above, methods of purification of GFPs, which can be used as FAMs, and luciferases, which can be used as BDPs, are known in the art.

Once prepared, the BDP and FAM are bridged together using chemical means. This is preferred if the modulator is not a peptide. If the modulator is a peptide a single entity recombinant-based method (see above) is often more amenable to effective production of the fusion molecule. A number of homo- hetero-bifunctional cross-linker reagents, all well known in the art, can be used to form the modulator (such as dithiobis-(succinimidyl propionate), N-(γ-maleimidobutyryloxy)succinimide, succinimidyl trans-4-(maleimidylmethyl)cyclohexane-1-carbxylate; see e.g. Pierce Catalog and Handbook and Molecular Probes' Handbook of Fluorecent Probes and Research Chemicals). Molecules and chemical groups forming the modulator or part of the modulator can be attached covalently to either the amino- or carboxy-terminus of the moieties, or to internally amino acids, as long as the activity of the desired activity of the fusion protein is obtained.

The modulator region or sequences can be formed during the attachment of the two moieties (e.g. FIG. 1) or added within the linker following the attachment of the BDP and the FAM using chemical means (e.g. FIG. 2; BDP-_{M}-FAM). In one embodiment, before their expression (by recombinant technology), purification and coupling, genes of the donor and acceptor moieties can be genetically modified to produce configurations as depicted in FIGS. 3b and 4b after their attachment. In another embodiment, after their expression (by recombinant technology) and purification but before their coupling, the BDP and FAM can be chemically modified to produce configurations depicted in FIGS. 3a and 4a. Configurations depicted in FIG. 3a and 4a can be also produced after the expression (by recombinant technology), purification and coupling of the donor and acceptor protein moieties (BDP and FAM) by chemically attached the modulator to one of the moieties. Finally, in each embodiment, the BDP and FAM can be modified prior their coupling in order to facilitate their attachment to each order. The chemical composition of the modulator or in some cases the linker, is not critical, however, the molecular distance between the BDP and FAM is important; it should be between 10 to 100 Å.

In another embodiment, the fusion molecule as depicted in FIG. 2 is produced by chemically fusing the isolated BDP and FAM together, thereby forming an non-peptide linker region in between the BDP and FAM (BDP-FAM or FAM-BDP). This fusion molecule is then modified by chemically or enzymatically attaching the modulator into the linker region as despite in FIG. 2.

Finally, the BRET fusion molecule having any of the configurations prepared herein can be used in an in vitro assay or an in vivo assay, using various techniques such as protein transfection techniques, permeabilizing agent, by micro-injection or electroporation to introduce the protein inside the cells. In each cases, the substrate is used to initiate the bioluminescent reaction (BRET assay). The substrate can be purchase from commercial suppliers (e.g. Molecular Probes), or synthesized.

Monitoring the Response of the BRET System

In a preferred embodiment, the energy transfer occurring between the donor (BDP) and acceptor (FAM) moieties is calculated from the emissions measured using optical filters (one for the FAM emission and the other for the BDP emission) that select specific wavelengths (see equation 1).

Ea/Ed=BRET efficiency (1)

where Ea is defined as the FAM emission intensity (emission light is selected using specific filter adapted for the emission of the acceptor) and Ed is defined as the BDP emission intensity (emission light is selected using specific filter adapted for the emission of the BDP).

It should be readily appreciated by those skilled in the art that the optical filters may be any type of filter that permits wavelength discrimination suitable for the BRET. For example, optical filters used in accordance with the present invention can be interference filters, long pass filters, short pass filters, etc. Intensities (usually in counts per second (CPS) or relative luminescence units (RLU)) of the wavelengths passing through filters can be quantified using either a photo-multiplier tube (PMT) or a CCD camera. The quantified signals are subsequently used to calculate energy transfer efficiencies. The energy transfer efficiency increases with increasing intensity of the acceptor emission. Generally, a ratio of the acceptor emission intensity over the donor emission intensity is determined (see equation 1), which is an abstract number that reflects energy transfer efficiency. The ratio increases with an increase of energy transfer (see Xu et al, (1999) Proc. Natl. Acad Sci. USA. 96, 151-156).

Energy transfer efficiencies can also be calculated using the inverse ratio of donor emission intensity over acceptor emission intensity (see equation 2). In this case, ratios decrease with increasing energy transfer efficiency. Prior to performing this calculation the emission intensities are corrected for the presence of background light and auto-luminescence of the substrate. This is correction is generally made by subtracting the emission intensity, measured at the appropriate wavelength, from a control sample containing the substrate but no BDP, FAM or fusion molecule.

Ed/Ea=BRET efficiency (2)

where Ea and Ed are as defined above.

The light intensity of the BDP and FAM emission can also be quantified using a monochromator-based instrument such as a spectrofluorometer, a charged coupled device (CCD) camera or a diode array detector. Using a spectrofluorometer, the emission scan is performed such that both BDP and FAM emission peaks are detected upon addition of the substrate. The areas under the peaks represent the relative light intensities and are used to calculate the ratios, as outlined above. Any instrument capable of measuring lights for the BDP and FAM from the same sample, can be used to monitor the BRET system of the present invention.

In an alternative embodiment the FAM emission alone is suitable for effective detection and/or quantification of BRET. In this case, the energy transfer efficiency is calculated using only the acceptor emission intensity. It would be readily apparent to one skilled in the art that in order to measure energy transfer, one can use the acceptor emission intensity without making any ratio calculation. This is due to the fact that ideally the FAM will emit light only if it absorbs the light transferred from the BDP. In this case only one light filter is necessary.

In a related embodiment the BDP emission alone is suitable for effective detection and/or quantification of BRET. In this case, the energy transfer efficiency is calculated using only the BDP emission intensity. It would be readily apparent to one skilled in the art that in order to measure energy transfer, one can use the donor emission intensity without making any ratio calculation. This is due to the fact that as the FAM absorbs the light transferred from the BDP there is a corresponding decrease in detectable emission from the BDP. In this case only one light filter is necessary.

In an alternative embodiment the energy transfer efficiency is measured using only one optical filter but still requires a ratiometric measurement. In this case, light intensity for the donor or the acceptor are determined using the appropriate optical filter and another measurement of the samples is made without the use of any filter (intensity of the open spectrum). In this latter measurement, total light output (for all wavelengths) is quantified. Ratio calculations are then made using either equation 3 or 4. For the equation 3, only the optical filter for the acceptor is required. For the equation 4, only the optical filter for the donor is required.

Ea/Eo−Ea=BRET efficiency or =Eo−Ea/Ea (3)

Eo−Ed/Ed=BRET efficiency or =Ed/Eo−Ed (4)

where Ea and Ed are as defined above and Eo is defined as the emission intensity for all wavelengths combined (open spectrum).

It should be readily apparent to one skilled in the art that further equations can be derived from equations 1 through 4. For example, one such derivative involves correcting for background light present at the emission wavelength for BDP and/or FAM.

In performing a BRET assay, light emissions can be determined from each well using the BRETCount. The BRETCount instrument is a modified TopCount, wherein the TopCount is a microtiterplate scintillation and luminescence counter sold by Packard Instrument (Meriden, Conn.). Unlike classical counters which utilise two photomultiplier tubes (PMTs) in coincidence to eliminate background noise, TopCount employs single-PMT technology and time-resolved pulse counting for noise reduction to allow counting in standard opaque microtiterplates. The use of opaque microtiterplates can reduce optical crosstalk to negligible level. TopCount comes in various formats, including 1, 2, 6 and 12 detectors (PMTs) which allow simultaneous reading of 1, 2, 6 or 12 samples, respectively. Beside the BRETCount other commercially available instrument are capable of performing BRET: the Victor 2 (Wallac, Finland (Perking Elmer Life Sciences)] and the Fusion (Packard Instrument, Meriden). BRET can be performed using readers that can detect at least the FAM emission and preferably two wavelengths (for the FAM and the BDP) or more.

To gain a better understanding of the invention described herein, the following examples are set forth. It should be understood that these examples are for illustrative purposes only. Therefore, they should not limit the scope of this invention in any way.

### EXAMPLES

### Example I

### Demonstration of an in vitro BRET Fusion Assay

A BRET assay using a fusion molecule is described wherein a Rluc moiety (BDP) is attached either genetically or chemically to an EYFP (FAM). A linker region is located between the two moieties that includes a specific protease cleavage site for enterokinase. Upon interaction of the enterokinase with the cleavage site in the sensor (Rluc-enterokinase-EYFP), the enzyme will cut the linker region, resulting in the separation of the Rluc and EYFP moieties and thereby causing an observable decrease of BRET (i.e. ratio 550/470 nm should decrease).

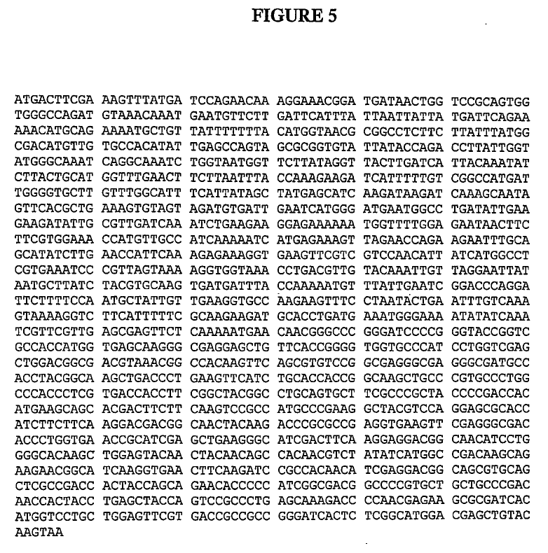

The DNA constructs used were as follows: the Rluc gene (pRL-CMV-Promega, Madison, Wis.), the Rluc:EYFP construct (FIG. 5; Xu, et al., 1999 Proc. Natl. Acad. Sci. USA, 96: 151-156), and the Rluc:enterokinase:EYFP construct (this construct is built by introducing DNA sequences coding for the enterokinase recognition site into the linker region of the Rluc:EYFP construct).

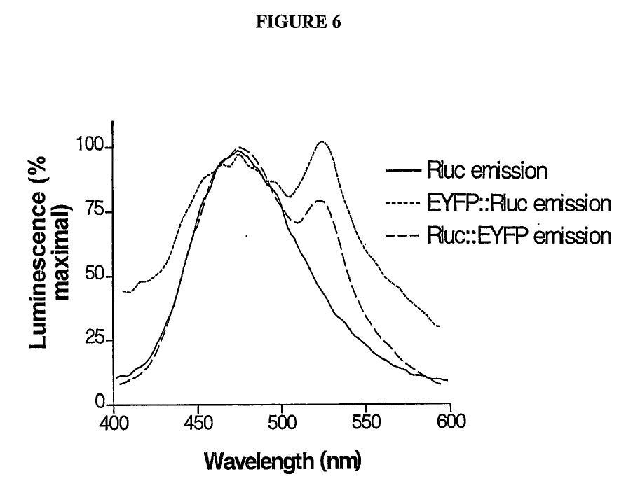

FIG. 6 shows the analysis obtained from cells transfected with pRL-CMV, pCDNA3.1/EYFP::Rluc and pCDNA3.1/Rluc::EYFP. Rluc (pRL-CMV) alone generated a typical emission peak centered at 475 nm. As described in Xu et al, (1999) Proc. Natl. Acad Sci. USA, 96, 151-156, Rluc::EYFP fusion protein generated two emission peaks at 476 nm and 525 nm corresponding respectively to Rluc and EYFP emission. The EYFP emission peak is generated by the transfer of energy between the two moieties. FIG. 41 shows that energy transfer also occurs between the Rluc and EYFP in the configuration EYFP::Rluc. An emission peak at 525 nm (corresponding to EYFP emission) is observed meaning that energy transfer occurred between the Rluc and the EYFP moieties. Note here that as for the configuration Rluc::EYFP, Rluc still the donor moiety in the EYFP:Rluc configuration. Therefore, this configuration can be used to introduce modulator element.

The amino acid sequence (Gly-Asp-Asp-Asp-Asp-Lys-Leu) for the enterokinase cleavage site was introduced into the linker region of Rluc:EYFP. The enterokinase enzyme recognizes the four Asp and the Lys amino acids and cuts the peptide linker after the amino acid, Lys.

The two complementary oligonucleotides (sense and antisense), corresponding to and used to introduce the enterokinase site (BRL/Gibco-Life Technologies, Gaethersburg, Md.), were annealed together using molecular biology techniques well known to those skilled in the art. The double strand of DNA was engineered to have cohesive ends (BamHI (5′ end) and KpnI (3′ end)) after annealing and was subcloned into the Rluc:EYFP construct at the BamHI-KpnI sites.

The DNA sequences of the complementary oligonucleotides, corresponding to the enterokinase site (sense and antisense) and having cohesive ends are as follows:

sense oligonucleotide: 5′ GATCCGGGCGACGATGACGATAAGTT GGCGGTA3′ (SEQ ID NO:9)

antisense oligonucleotide: 5′ CGCCAACTTATCGTCATCGTCGCCCG 3′ (SEQ ID NO:10)

The three of the above-mentioned DNA constructs were used to produce their corresponding proteins in bacteria, which proteins, in turn, were used as substrates for the enterokinase enzyme in the BRET assay (see below).

All three DNA constructs were subcloned in frame with the His tag sequences into the pQE plasmid (Qiagen, Mississauga, ON). This was done by introducing a His tag at the N-terminus of the constructs. The His tag allowed for the purification of different gene products from bacterial lysates using the Nickel (Ni) beads based technology (QIAexpress kits, Qiagen, Mississauga, ON). The constructs (Rluc gene alone, Rluc•EYFP and Rluc:enterokinase:EYFP) were subcloned into pQE-32 vector at the following restriction sites.

| | Construct | pQE-32 |
|---|---|---|
| Construct | restriction site | restriction site |
| Rluc | NheI*-XmaI | BamHI*-XmaI |
| Rluc::EYFP | NheI*-NotI* | SmaI |
| Rluc:enterokinase:EYFP | NheI*-NotI* | SmaI |

*all these sites were blunt ended before being subcloned into the pQE-32 plasmid, using Klenow enzyme under standard conditions and using molecular biology techniques known to those skilled in the relevant art (Ausubel, F. M. et al., Current protocols in molecular biology, Vol. 1 (1995) John Wiley & Sons, Inc., and Sambrook, J. et al., Molecular Cloning: A laboratory manual, 2^{nd} ed. (1989) Cold Spring Harbor Laboratory Press).

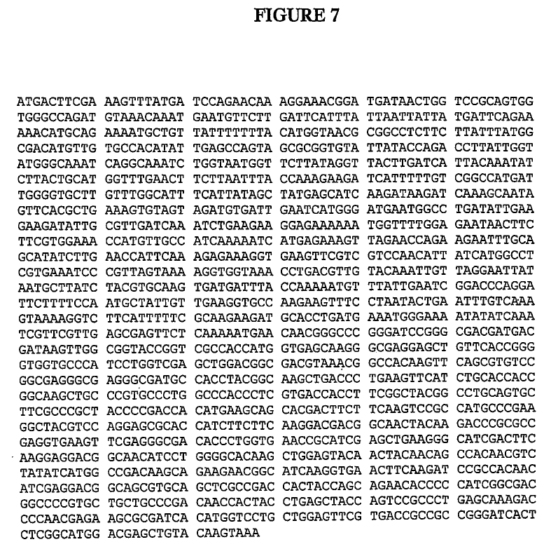

The DNA sequences for the two fusion constructs (SEQ ID NOs: 1 and 2, respectively) of this example are presented in FIGS. 6 and 7. The Rluc DNA sequence can be found in GeneBank under the accession number: M63501

The DNA constructs in the pQE-32 vectors were then transfected into suitable bacteria (M15) for protein expression. Bacteria were inoculated in 100 ml of LB media and grown for 4 hours at 37° C. until they reached a density of 0.6 OD₆₀₀. Protein expression was induced by adding IPTG (1 mM) into the bacterial cultures for 16 hours at 37° C. The different proteins were purified from the bacterial lysates using 0.5 ml Ni-NTA resin as described by a standard manufacturer's protocol (Qiagen, Mississauga, ON). The purified proteins were then desalted and concentrated (to a volume between 400-700 μl) using a Centriprep-10 or -30 and Centricon-10 or 30 (Amicon, Beverly, Mass.) with a PBS+NaCl 250 mM buffer. Protein concentrations were determined by the Bradford assay (Bradford, M. M., 1976, Anal. BioChem. 72: 248-254) using bovine serum albumin as a standard. Once purified, the proteins were used in the enterokinase BRET assay. The purity of the extracted protein was determined by SDS-PAGE to be between 60-80%.

The BRET assay was performed by mixing 60-100 μg of purified protein for each well with the BRET assay buffer (PBS+Ca²⁺1 mM Mg²⁺0.5 mM+glucose (1 g/l)+10 mM DTT) to a total volume of 150 μl. Each assay was conducted in triplicate (i.e. three wells) in 96-well Optiplate plate (Packard, U.S.A.). Half of the assays were done in the presence of two units of the enterokinase enzyme (Invitrogen, Carlsbad, Calif.). Coelenterazine h (Molecular Probes, Eugene, Oreg.) at a concentration of 20 μM/50 μl was added to each well to start the bioluminescent reaction of Rluc. The final concentration of coelenterazine h was 5 μM. The total volume of the assay was 200 μl per well. After the addition of the coelenterazine h, the microtiterplates were loaded into the BRETCount. Detection was performed using the BRETCount at 30° C. for four hours. For each time point and assay, ratios were calculated by dividing the emission light output at 550 nm by the emission light output at 470 nm.

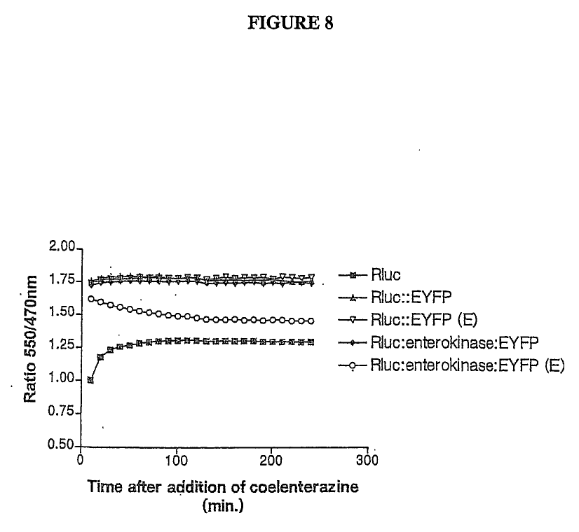

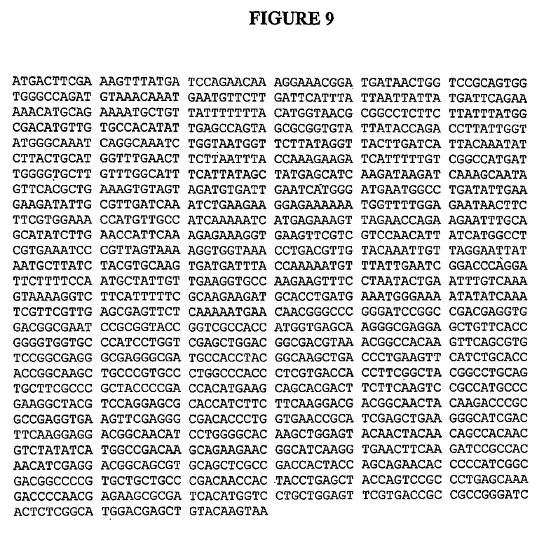

Only the ratio for the construct Rluc:enterokinase:EYFP in the presence of the enterokinase enzyme decreased over time (FIG. 8) demonstrating that more and more constructs were cut. The ratios from the negative controls (Rluc and Rluc:EYFP) did not change over time when in the presence or absence of the enterokinase enzyme.

The assay results demonstrated that BRET can be carried out with partially purified proteins. This example is also an illustration of the BRET assay using a fusion protein, wherein a specific amino acid sequence can be inserted in between the Rluc and EYFP moieties, such as a protease site (or in the alternative, a phosphorylation site, or a site that recognizes analytes e.g. Ca²⁺).

The TopCount is a microtiterplate scintillation and luminescence counter sold by Packard Instrument (Meriden, Conn.). Unlike a classical counter which utilized two photomultiplier tubes (PMTs) in coincidence to reduce noise, TopCount employs a single-PMT technology and time-resolved pulse counting for noise reduction to allow counting in an opaque standard format microtiterplate. The use of opaque microtiterplates can reduce optical crosstalk to negligible levels. The TopCount comes in various formats (eg. with 1, 2, 6, 12 detectors (PMTs)), which can read 1, 2, 6 or 12 samples simultaneously depending on the format.

A modified 2-detector TopCount (BRETCount) was used to measure BRET in this example. Modifications included positioning the bandpass interference filters under each TopCount PMT (between the PMT and the detector mounting plate), and adjusting the TopCount software to allow each PMT to read each well of a microtiterplate. These changes allowed the sequential detection of light emitted at specific wavelengths emitting from the samples. Accordingly, for each well (or sample) we obtained two light output values corresponding to each filter. Beside its throughput (microtiterplate reader), the TopCount is a temperature controlled instrument that is useful when performing cell-based assays or timecourse experiments.

The two filters used were 1) for the acceptor emission (fluorophore): 550DF80 (Omega Optical Inc, Brattleboro Vt.); and 2) for the donor emission (bioluminescent protein): 470DF60 (Omega Optical Inc, Brattleboro Vt.).

BRET is measured by dividing the light output for the 550 nm filters by the 470 nm light output (acceptor/donor). The instrument parameters used for the BRETCount were luminescence SPC mode and at a read length of 1 sec for each PMT.

### Example II

### Apoptosis Sensor

Apoptosis or PCD (programmed cell death) is a normal cellular process found in most cell types. It is involved in many aspects of cell homeostasis and organism development (e.g. PCD is responsible of the modeling (shape) of an organ during morphogenesis) (Nicholson, D. W. and Thornberry, N. A., 1997, TIBS, 22: 299-306; Kinloch, R. A., et al., 1999, TIPS, 20: 35-42). Very often, inappropriate PCD is found in many diseases like cancer, neurodegenerative diseases, etc. PCD is a regulated way for cells to die at a specific time without stimulating the defense mechanism of the organism (e.g. inflammation) and hence distinct from the other type of cell death, necrosis, generally resulting from cellular injury.

Many proteins and lipids are involved in PCD. Among them, the caspases which are part of a large family of cysteine proteases (Nicholson, D. W. and Thornberry, N. A., 1997, TIBS, 22: 299-306). Caspases recognize and cleave specific amino acid sequences (usually a sequence of 4-5 amino acids) and are part of a protein cascade that ends with the activation of effectors involved in the degradation of protein (by activating general proteases) and DNA. Specific receptors at the plasma membrane e.g. fas-R, TNF-R) and their adaptor proteins trigger the apoptotic signal by the activation of a first line of caspases (caspase-2, -8, -10) depending of the cell type). These activated caspases then activate other caspases (-3, -7, -9, -4) by cleaving a specific amino acid sequence. These, other caspases, in turn, may activate still other caspases and also act on cellular targets to generate the cell disruption. Apoptosis can be triggered also by chemical agents in some cell types (e.g. thapsigargin on the HL-60 cell line, or staurosporine on the HeLa cell line).

A BRET apoptosis sensor was engineered by introducing a caspase-3 recognition site in the linker region of the Rluc-EYFP fusion construct. Upon induction of apoptosis, caspase-3 recognizes and cleaves the linker region, thereby separating Rluc from EYFP and thereby decreasing the BRET ratio over time.

The caspase-3 site was introduced using annealed complementary oligonucleotides. The two complementary oligonucleotides used encode the caspase-3 site: Gly-Asp-Glu-Val-Asp-Gly. Only a Asp-Glu-Val-Asp sequence is needed for recognition by caspase-3. Caspase-3 cuts between the Asp and Gly residues. The DNA sequences of the oligonucleotides used are as follows:

Sense oligonucleotide: 5′ GATCCGGCCGACGAGGTGGACGGCGAA TCCGCGGTAC 3′ (SEQ ID NO:11)

Antisense oligonucleotide: 5′ CGCGGATTCGCCGTCCACCTCGTCGGCCG 3′ (SEQ ID NO:12)

Once annealed, the oligonucleotides formed a double strand of DNA having cohesive restriction sites at each of the ends: 5′ BamHI and at 3′ KpnI. pT7/Rluc-EYFP was digested using BamHI and KpnI and the annealed double oligonucleotide was subcloned into the pT7/Rluc-EYFP vector. The Rluc-EYFP and the Rluc-caspase-EYFP fusion genes were then subcloned from the pT7 vector into the mammalian expression vector, pCEP4 (Invitrogen) using NheI and NotI restriction enzymes.

The DNA sequences of the two constructs needed in this example are presented in FIGS. 5 (SEQ ID NO: 1) and 9 (SEQ ID NO: 3).

pCEP4/Rluc-EYFP and pCEP4/Rluc-caspase-EYFP DNAs were transfected into HeLa cell using LipofectAMINE (BRL/Gibco-Life technologies) in 100 mm dishes as described in the manufacturer's protocol. 24 hours post-transfection, transfected cells were harvested and distributed into 96-well microtiterplates (white Optiplate from Packard) at a density of 30,000 cells per well. The following day, cells were induced to undergo apoptosis using 1 μM staurosporine dissolved in ISCOVE media (BRL/Gibco-Life technologies) without serum and phenol red at 37° C. (total volume here is 100 μl) for 5 hours. 50 μl of BRET buffer (PBS Ca²⁺/Mg²⁺+glucose without aprotinin) and 50 μl of coelenterazine h (20 μM) were added to start the bioluminescence reaction. Light emissions were detected using the BRETCount (read length 1 sec for each filter, temperature 25° C.). Data was collected 10 minutes after the addition of coelenterazine h. The assay was done in quadruplicate.

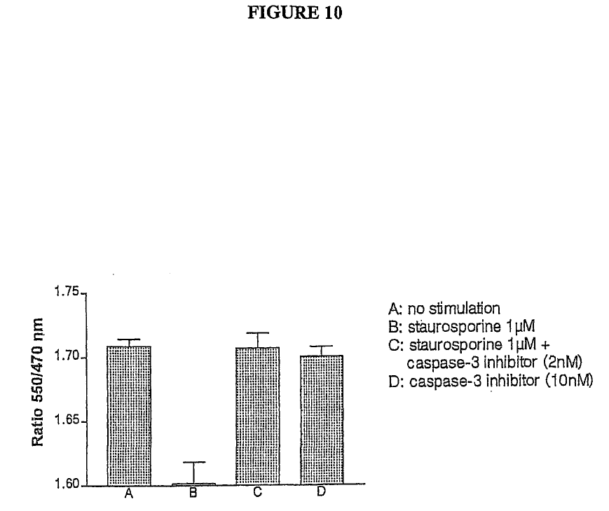

FIG. 10 gives representative BRET ratio changes in HeLa cells after 5 hours of apoptosis induction by staurosporine. A significant ratio change (0.1 unit) occurs when cells transfected with the apoptosis sensor were induced by staurosporine. Ratios decreased.

### Example III

### Alternative Apoptosis Sensor

In this example, a BRET apoptosis sensor was prepared by introducing a caspase-3 recognition site in the linker region of the GFP-Rluc fusion construct. Upon induction of apoptosis, caspase-3 recognizes and cleaves the linker region, thereby separating Rluc from EGFP. Therefore, induction of apoptosis will decrease the BRET ratio over time.

pGFP1::Rluc Construct

pGFP1::Rluc was made by introducing the codon humanized Rluc gene from the pCDNA3.1/Rluc (h) vector into the pGFP1-C2 vector (see Technical data sheet from BioSignal Packard, Montreal). In order to prepare pCDNA3.1/Rluc (h) the cDNA of Rluc was codon humanized using methods previously described (U.S. Pat. No. 5,874,304 and Zolotukhin et al, J. Virol. 70: 4646-4654, 1996). Briefly, humanized Rluc (hRluc) was synthesized using a series of ligated polymerase extended oligonucleotides. The hRluc sequence confirmed by DNA sequencing and hRluc was subcloned in pCDNA3.1 zeo (+) to form pCDNA3.1/Rluc (h).

GFP1 is a mutant of the Green Fluorescent Protein (GFP) having a unique mutation at position 64 (amino acid positioning is relative to the GFP wild type) where the phenylalanine has been replaced by a leucine. pCDNA3.1/Rluc (h) was digested with ApaI and BamHI. After digestion, products were separated on an agarose gel. A band corresponding to Rluc (h) gene (around 920 base pair in length) was cut from the agarose gel and purified using the Qiaquick spin kit (Qiagen). The purified band was then subcloned in the pGFP1-C2 vector digested with ApaI and BamHI. The linker between GFP1 and Rluc was then shortened by digesting the pGFP1::Rluc vector with BspE1 followed by a fill-in reaction using Klenow enzyme to blunt the end. After the Klenow reaction, the product was purified using Qiaquick spin kit, digested with EcoRV, purified another time (Qiaquick) and then ligated using ligase. This procedure removed 47 nucleotides from the original pGFP1-C2 vector and creates the following linker between GFP1 and Rluc:

| | |
|---|---|
| TCCGGATCAAGCTTGCGGTACCGCGGGCCCTCT | (SEQ ID NO:13) |
| AGAGCCACCATG. | |

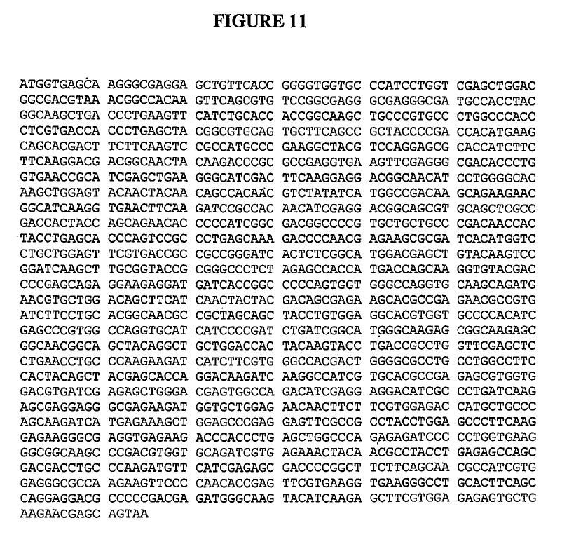

This linker contains convenient unique restriction site that can be used to subclone fragments between the GFP1 and Rluc genes. These restriction sites are (5′ to 3′ orientation): BspE1, HindIII, KpnI, SacII, ApaI and XbaI. FIG. 11 shows a DNA sequence (SEQ ID NO:4) encoding the GFP:Rluc fusion protein containing a unique 14 amino acid linker region between the GFP and the Rluc.

Introduction of Caspases-3 Site between GFP1::Rluc Construct

The caspase-3 site was introduced in the pGFP1::Rluc vector using annealed complementary oligonucleotides. We annealed two complementary oligonucleotides encoding the following caspase-3 site: Gly-Asp-Glu-Val-Asp-Gly. Only the sequence Asp-Glu-Val-Asp is needed for recognition by caspase-3. Caspase-3 cuts between the Asp and Gly residues.

Sense oligonucleotide: 5′ AGCTTGGGCGACGAGGTGGACGGCGGGCC 3′ (SEQ ID NO:14)

Antisense oligonucleotide: 5′ ACCCGCTGCTCCACCTGCCGC 3′ (SEQ ID NO:15)

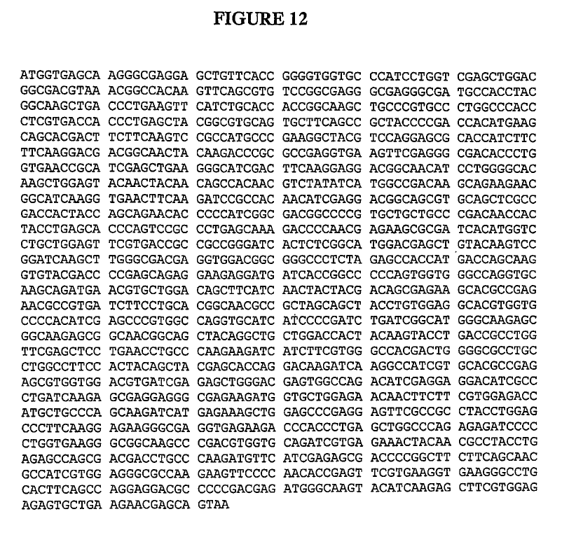

The above two oligonucleotides (sense and antisense) were synthesized (by BRL/Gibco-Life Technologies). The two oligonucleotides are complementary to each other and were annealed together using standard molecular biology techniques (Ausubel, F. M. et al.: Current protocols in molecular biology, Vol. 1 (1995) John Wiley & Sons, Inc.; Sambrook, J. Fritsch, E. F. & Maniatis, T.: Molecular Cloning: A laboratory manual, 2^{nd} ed. (1989) Cold Spring Harbor Laboratory Press). The oligonucleotides were engineered in order to generate cohesive ends after their annealing. HindIII is found at the 5′ end and ApaI is found at the 3′ end. The double stranded product generated after annealing was introduced into the GFP1::Rluc construct digested using HindIII and ApaI. HindIII and ApaI are unique restriction sites found in the linker region between the GFP and Rluc genes. The final structure is GFP1:caspase-3:Rluc and as shown in FIG. 12. Since originally the GFP::Rluc was already in a expression vector compatible for transfection and expression of the protein in mammalian cells (pGFP background), no further modification was required. The pGFP1:caspase-3:Rluc plasmid DNA used for the transfections was purified using the Maxi-Prep kit from Qiagen.

Expression

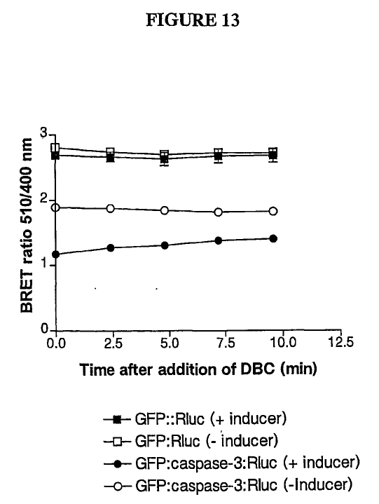

PGFP1:caspase-3:Rluc DNAs and the original plasmid pGFP1::Rluc (without the caspase-3 site) were transfected into HeLa cell using LipofectAMINE (BRL/Gibco-Life technologies) in 100 mm dishes and 8 μg of plasmid DNA as described in the manufacturer's protocol. Twenty-four hours post-transfection transfected cells were harvested and distributed into 96-well microtiterplate (white Optiplate from Packard) at a density of 30 000 cells per well. The following day, cells were washed with PBS and were induced to undergo apoptosis by adding 100 μl of Iscove media without serum and phenol red (BRL/Life Technologies) containing staurosporine at a final concentration of 1 μM (+inducer; see FIG. 13) or 100 μl of Iscove alone (-inducer). Cells were incubated for 5 hours at 37° C. After this period, 50 μl of BRET buffer (PBS Ca²⁺/Mg²⁺+glucose without aprotinin) and 50 μl of DeepBlueC (20 μM) were added to start the bioluminescence reaction. Total assay volume was 200 μl. Light emissions were detected using the BRETCount (read length of 1 sec. for each filter, SPC mode, temperature 25° C.). The results represent the mean±SEM of quadruplicate wells. The graphs were generated using GraphPad PRISM software. In all cases, background levels (wells with only DBC in PBS+) were subtracted.

Results

FIG. 13 represents the ratio changes in HeLa cells after 5 hours of apoptosis induction by staurosporine. A significant BRET ratio change (0.72 unit at time zero) occurred when cells were transfected with the apoptosis sensor (pGFP1:caspase-3:Rluc) and then induced to undergo apoptosis by staurosporine. As expected, the ratio decreased indicating that cellular caspase-3 recognized and cut the cleavage site between the GFP1 and Rluc moieties. No change in BRET ratio was observed in cells transfected with the pGFP::Rluc or in cells transfected with the pGFP:caspases-3:Rluc without staurosporine (-inducer).

The: GFP1::Rluc construct was sequenced in its entirety (SEQ ID NO:4) using an ABI automated sequencer (ABI Prism 310 Genetic Analyzer from Perkin Elmer).

### Example IV

### BRET Assay Using a Fusion Molecule Sensitive to Phosphorylation

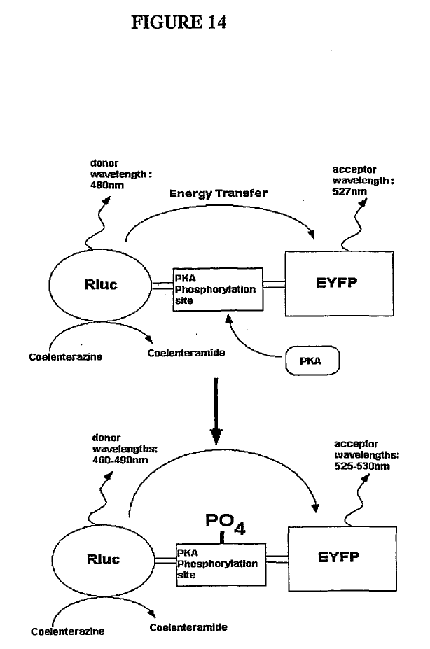

This example demonstrates the effect on the energy transfer between the donor and the acceptor of the presence of a phosphate group in the modulation region (see FIG. 14). The Rluc::EYFP fusion molecule has been used to make another fusion called Rluc:PKA:EYFP in which a phosphorylation site for protein kinase A (PKA) has been introduced in between the donor and acceptor moieties.

DNA Construct

The pT7/Rluc::EYFP vector DNA (from Xu et al, (1999) Proc. Natl. Acad. Sci. 96, 151-156) was digested with NheI-NotI to release the Rluc::EYFP insert (approximately 1.7 kB). This insert was then subcloned into pCDNA3.1/zeo (+) using the NheI and NotI restriction sites to form a new vector called pCDNA3.1/Rluc::EYFP. This new vector was digested at unique BamHI and KpnI sites found inbetween the Rluc gene and EYFP gene. A chemically synthesized DNA insert encoding SEQ ID NO: 16 was directionally introduced inbetween the digested sites to form the final pCDNA3.1/Rluc:PKA:EYFP plasmid.

The PKA phosphorylation site introduced in between Rluc and EYFP genes (Rluc::EYFP) was: LRRASLG (single amino acid code, SEQ ID NO:16) based on the sequence found in Kemp, B. E. et al. (1977) J. Biol. Chem. 252, 4888. The cellular PKA enzyme recognizes and phosphorylates the serine residue. The overall modulator region is 18 amino acids in length.

Two oligonucleotides (sense and antisense) were synthesized (by BRL/Gibco-Life Technologies) corresponding to the PKA site. The two oligonucleotides are complementary to each other and were annealed together using standard molecular biology techniques. The oligonucleotides were engineered in order to generate cohesive ends after annealing, ie. BamHI (5′ end) and KpnI (3′ end), so that the double stranded DNA, could be subcloned into the Rluc::EYFP at the BamHI-KpnI sites.

The DNA sequences of the complementary oligonucleotides corresponding to the PKA site and having the cohesive ends are as follows:

sense oligonucleotide 5′ GATCCGCTGAGGAGGGCCAGCCTGGGCGC GGTAC 3′ (SEQ ID NO:17)

antisense oligonucleotide: 5′ CGCGCCCAGGCTGGCCCTCCTCAGCG 3′ (SEQ ID NO:18)

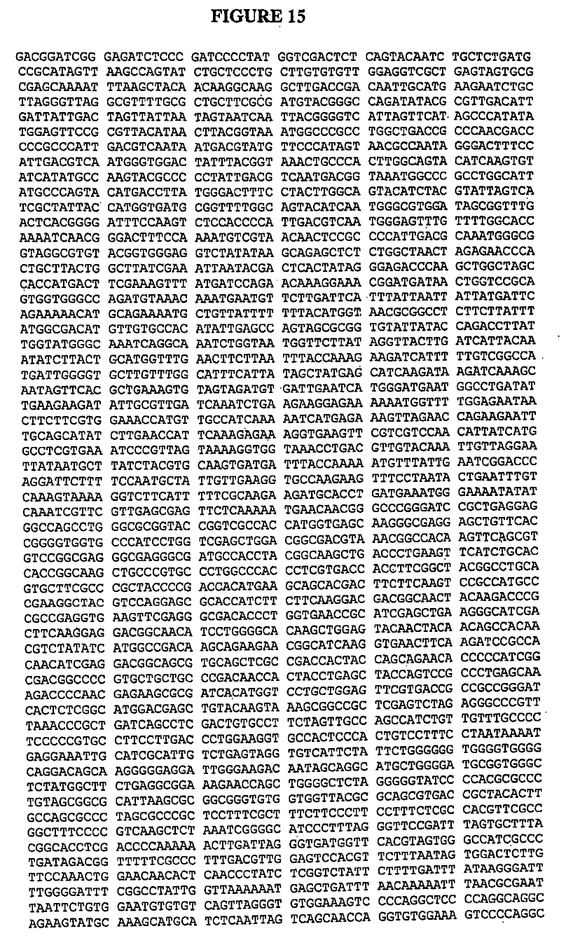

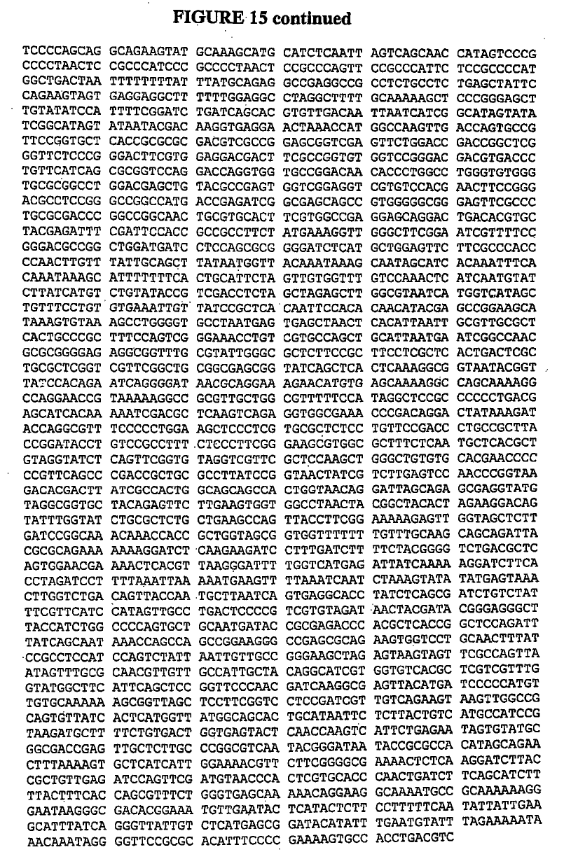

The DNA sequence for the Rluc:PKA:EYFP fusion construct is shown in FIG. 15.

Transfection and BRET Assay.

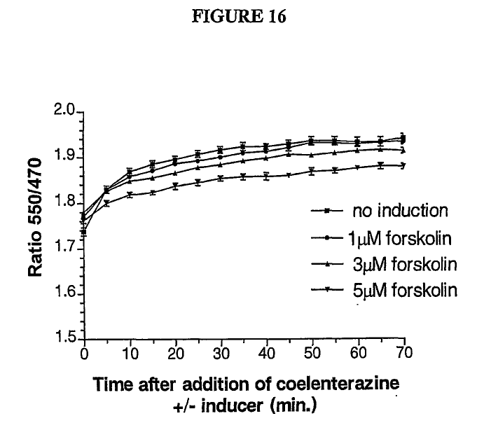

CHO-K1 cells were transfected using the pCDNA3.1/Rluc:PKA:EYFP plasmid and LipofectAMINE reagent in 100 mm dishes (0.2 μg DNA) according to the manufacturer's protocol (Life Technologies, Rockville, Md.). Two days after transfection, transfected cells were starved for two 2 hours in MEM before being washed (in PBS), harvested, counted and distributed into 96-well microtiterplate (white Optiplate from Packard Instruments, Meriden, Conn.) at a density of 50 000 cells per well in PBS (Life Technologies Cat. No. 14287-080)+10 mM DTT and 2 μg/ml aprotinin. Cellular PKA enzyme is activated by increasing the cAMP content of the cell. This can be achieved using the forskolin drug which is an activator of adenylate cyclase. Adenylate cyclase is a integral membrane protein producing cAMP from ATP once activated by G-proteins or drug such as forskolin. Therefore, forskolin was added to each well at various concentrations (see FIGS. 16). Coelenterazine (the h derivative from Molecular Probes, Eugene, Oreg.) to a final concentration of 5 μM was then added to start the bioluminescence reaction of the Rluc. The final assay volume per well was 200 μl. Light emissions from each well were quantified using the BRETCount. The BRETCount instrument is a modified TopCount. The TopCount is a microtiterplate scintillation and luminescence counter sold by Packard Instrument (Meriden, Conn.). Unlike the classical counters which utilized two photomultiplier tubes (PMTs) in coincidence to reject noise, TopCount employs single-PMT technology and time-resolved pulse counting for noise reduction to allow counting in an opaque standard format microtiterplate. The use of opaque microtiterplates can reduce optical crosstalk to negligible levels. The TopCount comes in various formats: eg. 1, 2, 6, 12 detectors (PMTs) which can read simultaneously 1, 2, 6 or 12 samples, respectively.

We have modified a 2-detector TopCount in order to measure BRET. This prototype was later called BRETCount. Bandpass interference filters were positioned under each TopCount PMT (between the PMT and the detector mounting plate). Furthermore, the TopCount software was modified to allow each well of a microtiterplate to be read by each PMT. These modifications allow the sequential detection of light emitted at specific wavelengths from the samples. Therefore, for each well (or sample) we obtained two light output values corresponding to each filter. Beside its throughput (microtiterplate reader), the TopCount is a temperature controlled instrument which is useful when performing cell-based assays or timecourse experiments.

The bandpass interference filters were designed based on emission spectra. The two filters used were:

for the acceptor emission (fluorophore): 550DF80 (Omega Optical Inc, Brattleboro Vt.)

for the donor emission (bioluminescent protein): 470DF60 (Omega Optical Inc, Brattleboro Vt.)

In this example, BRET was measured by dividing the light output for the 550 nm filters by the 470 nm (acceptor/donor). Instrument parameters used for the BRETCount were:

Assay temperature: 30° C.

Single Photon Counting mode

Read length of 1 sec for each PMT

Results

FIG. 16 represents the effect of forskolin on the BRET ratio (550/470) over time. At any specific time after addition of coelenterazine, the use of forskolin decreases the BRET ratio indicates that the energy transfer is perturbed (decreased) by the presence of the phosphate group added by PKA to the site positioned in between the Rluc and EYFP moieties. Furthermore, the change in the BRET ratio is forskolin dose-dependent such that the BRET ratio decreases with an increase in the concentration of forskolin.

### Example V

### Preparation of a Fusion Molecule with a Modulator Containing a Kemptide Sequence

A) Modulator-FAM-BDP Configuration

Molecular Biology

1. Introduction of the GFP1::Rluc Fusion Gene into the pQE60 Vector

The pQE60 vector is bacterial expression vector commercialized by Qiagen. Any DNA fragment when subcloned into the multiple cloning site of pQE60 and expressed, is fused to a His-tag sequence at its C-terminus; this vector is used to express a gene of interest fused to a His-tag, in bacteria. Once the fusion protein has been expressed, the His-tag sequence is used to purify the fusion protein using Ni-NTA bead technology (see the Qiaexpressionist booklet July 1998 Third Ed. from Qiagen). In order to incorporate the DNA sequence encoding the fusion GFP1::Rluc construct into pQE60, we removed the Rluc and GFP stop codons. This was done by PCR (loop out) using primers that were designed to omit the stop codons during amplification. The PCR also primers contained unique restriction sites to facilitate the subcloning of the amplified products. The primers were also engineered in order for the GFP gene, the Rluc gene and the His-tag to all be in frame. The DNA sequences of the PCR primers used to amplify the GFP1 gene without its stop codon are:

Sense primer: 5′ CATGCCATGGGCCACCATGGTGAGCAAGG 3′ (the NcoI restriction site is underlined) (SEQ ID NO:19)

Antisense primer: 5′ CGGGATCCGGACTTGTACAGCTC 3′ (the BamHI restriction site is underlined) (SEQ ID NO:20)

The DNA sequences of the PCR primers used to amplify the Rluc gene without its stop codon were:

Sense primer: 5′ CGGGATCCAGCTTGCGGTACCGCGGGCCCTCTAGAGCC ACCATGACTTCGAAAGTT 3′ (the BamIII restriction site is underlined) (SEQ ID NO:21)

Antisense primer: 5′ GAAGATCTTTGTTCATTTTTGAGAACTCGC 3′ (the BglII restriction site is underlined) (SEQ ID NO:22)

2. PCR Amplification

PCR was performed in 50 μl using a 9600 PCR Turbo instrument from Perkin Elmer. The pBRET+vector (see technical data sheet from BioSignal Packard) was used as the DNA template for the PCR. This vector contains a fusion Rluc::GFP1 gene construct. The PCR primers and dNTP were ordered from Life technologies. The PFU turbo polymerase from Stratagene was used for the amplification. PCR reactions were carried out as follows:

Denaturation step at 95° C. for 2 min.

Hot start at 80° C.

25 cycles of: denaturation 95° C. 1 min.

annealing 58° C. for 10 sec for GFP amplification and at 56° C. for the Rluc amplification

elongation 72° C. for 1 min.

Final elongation step 72° C. for 10 min

Both the amplified Rluc and GFP PCR products were subcloned first into the PCR TOPO Blunt II vector (Invitrogen) according to the manufacturer's protocol which was then used to transform standard, competent (bacterial) cells (Ausubel, F. M. et al.: Current protocols in molecular biology, Vol. 1 (1995) John Wiley & Sons, Inc.; Sambrook, J. Fritsch, E. F. & Maniatis, T.: Molecular Cloning: A laboratory manual, 2^{nd} ed. (1989) Cold Spring Harbor Laboratory Press). Positive clones were screened by restriction digests with BamHI, BglII and NcoI enzymes. One positive clone for the GFP was chosen and digested with NcoI and BamHI restriction enzymes to excised the the DNA fragment corresponding to the amplified GFP (around 720 base pair in length), which was then separated out on an agarose gel, cut out of the gel and purified using the QiaQuick kit (Qiagen). This fragment was then subcloned into the multiple cloning site of pQE60 vector using the NcoI and BamHI restriction sites. The pQE-60 vector was first digested with NcoI and BamHI restriction enzymes and then dephosphorylated with CIAP using a standard protocol (Ausubel, F. M. et al.: Current protocols in molecular biology, Vol. 1 (1995) John Wiley & Sons, Inc.; Sambrook, J. Fritsch, E. F. & Maniatis, T.: Molecular Cloning: A laboratory manual, 2^{nd} ed. (1989) Cold Spring Harbor Laboratory Press). The linearized vector was then purified using agarose gel electrophoresis and a Qiaquick kit (Qiagen). Positive clones indicative of the insertion of the GFP DNA fragment into the pQE60 vector were screened using a NcoI/BamHI double digest.

A positive clone was chosen and digested using BamHI and BglII enzymes and then purified using Qiaquick kit (Qiagen). This linearized vector was used for the subcloning of the amplified Rluc. One positive Rluc clone (from the PCR TOPO blunt II vector) was chosen and digested with BamI and BglII enzymes. The resulting DNA fragment corresponding to the amplified Rluc (around 9230 base pair in length) was separated out on agarose gel, cut out of the gel and purified using the QiaQuick kit (Qiagen). This fragment was then subcloned into the pQE60/GFP vector at the BamHI and BglII restriction sites to yield the pQE60/GFP1::Rluc vector. The GFP1::Rluc construct in pQE60/GFP1::Rluc was sequenced in its entirety using an ABI automated sequencer (ABI Prism 310 Genetic Analyzer from Perkin Elmer).

3. Introduction of the Kemptide Sequence at the N-terminus of the GFP1::Rluc

The Kemptide sequence is the amino acid sequence LRRASLG (in single amino acid code) which is recognized and phosphorylated by protein kinase A (PKA) on the serine residue. This sequence is often used as peptide substrate to measure PKA activities in cell extracts (Giembycz et al. (1990) Biochem, Pharmacol. 39, 271-283; Langlands et al. (1990) Biochem Pharmacol. 39, 1365-1374. Kemptide sequence has been introduced at the beginning of the fusion GFP1::Rluc construct.

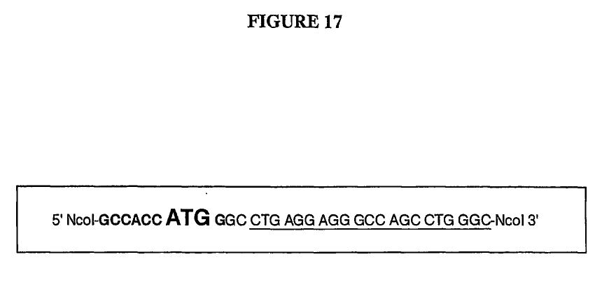

Two oligonucleotides (sense and antisense) were synthesized (by BRL/Gibco-Life Technologies) encoding the kemptide sequence. The two oligonucleotides are complementary to each other and were annealed together using standard molecular biology techniques (Ausubel, F. M. et al.: Current protocols in molecular biology, Vol. 1 (1995) John Wiley & Sons, Inc.; Sambrook, J. Fritsch, E. F. & Maniatis, T.: Molecular Cloning: A laboratory manual, 2^{nd} ed. (1989) Cold Spring Harbor Laboratory Press). The oligonucleotides were engineered in order to generate cohesive ends after their annealing. A methionine residue has been positioned before the kemptide sequence in order for the final construct Kemptide:GFP1::Rluc fusion construct to be correctly and efficiently translated. Furthermore, a glycine residue has been added next to the first methionine in order to create a perfect Kozak consensus (see FIG. 17 for an overview of the modulator). The two cohesive ends were made of NcoI recognition/cleavage sequence. Hence, after the annealing step, the double stranded DNA was subcloned into the GFP1::Rluc (of pQE60/GFP1::Rluc) using the NcoI site located at the beginning of the GFP1::Rluc construct. The modulator sequence has been engineered in order to be in frame with the GFP::Rluc sequences.

The DNA sequences of the oligonucleotides corresponding to the kemptide sequence and having cohesive ends are as follows:

Sense oligonucleotide: 5′ CATGGGCCACCATGGGCCTGAGGAGGGCCAG CCTGGGCC 3′ (SEQ ID NO:23)

Antisense oligonucleotide: 5′ CCGGTGGTACCCGGACTCCTCCCGGTCGGA CCCGGGTAC 3′ (SEQ ID NO:24)

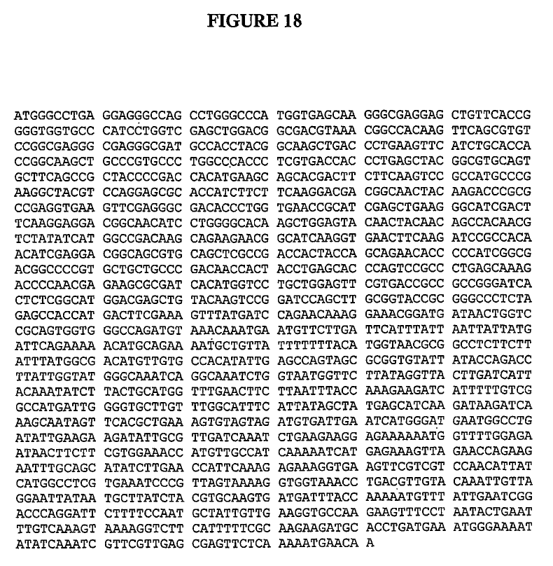

After subcloning, the modulator:GFP::Rluc construct was sequenced in its entirely using an ABI automated sequencer (ABI Prism 310 Genetic Analyzer from Perkin Elmer). The DNA sequence of the final fusion product in presented in FIG. 18.

Note here that the order of insertion of the various components into the final need not be done in the order described above. For example, the modulator can be inserted first and then the fusion gene GFP::Rluc inserted to create the final construct.

B) FAM-BDP-Modulator Configuration

In this configuration, a kemptide sequence was introduced after the coding region of Rluc in the fusion GFP1:Rluc protein construct.

Two oligonucleotides (sense and antisense) were synthesized (by BRL/Gibco-Life Technologies) corresponding to the kemptide sequence. The two oligonucleotides are complementary to each other and were annealed together using standard molecular biology techniques (Ausubel, F. M. et al.: Current protocols in molecular biology, Vol. 1 (1995) John Wiley & Sons, Inc.; Sambrook, J. Fritsch, E. F. & Maniatis, T.: Molecular Cloning: A laboratory manual, 2^{nd} ed. (1989) Cold Spring Harbor Laboratory Press). The oligonucleotides were engineered in order to generate cohesive ends after annealing. The two cohesive ends consisted of the BglII recognition/cleavage sequence. After annealing the double stranded DNA was subcloned at the BglII site located at the end of the GFP1::Rluc construct. The modulator sequence was engineered to be in frame with the GFP1::Rluc sequences

The DNA sequences of the complementary oligonucleotides corresponding to the kemptide sequence and having the cohesive ends are as follows:

Sense oligonucleotide: 5′ GATCTCTGAGGAGGGCCAGCCTGGGCA 3′ (SEQ ID NO:25)

Antisense oligonucleotide: 5′ AGACTCCTCCCGGTCGGACCCGTCTAG 3′ (SEQ ID NO:26)

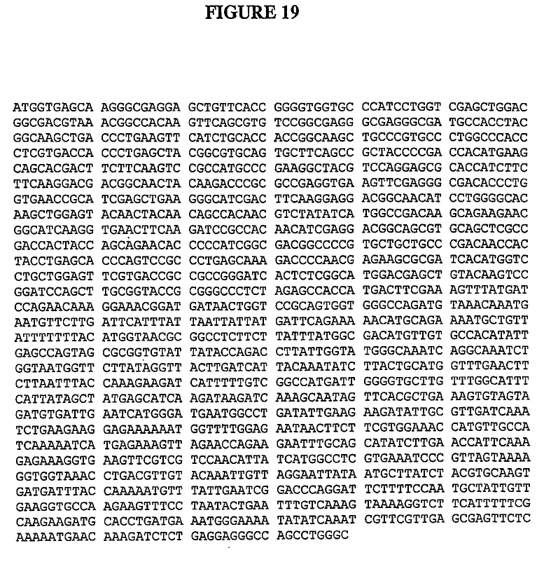

After subcloning, the: GFP::Rluc:modulator construct was sequenced in its entirety using an ABI automated sequencer (ABI Prism 310 Genetic Analyzer from Perkin Elmer). The DNA sequence of the final fusion product in presented in FIG. 19.

C) Other Strategy for Subcloning the Modulator.

The modulator (in this case the kemptide sequence) in the alternative can be introduced using the polymerase chain reaction (PCR). In this approach, the kemptide sequence is incorporated into one of the primers used for the PCR. Where the modulator sequence is positioned at the beginning of the fusion protein (modulator:GFP::Rluc), the primers (oligonucleotides) could have the following structure:

Sense primer (5′-3′ orientation): Restriction site-methionine-kemptide sequence-Rluc or GFP coding region

Antisense primer: (5′-3′ orientation): Restriction site-stop codon-Rluc or GFP coding region).

If the modulator is positioned at the end of the fusion construct (GFP::Rluc:modulator), then the reverse (antisense) primer would contain the DNA sequence encoding for the Kemptide sequence.

Note that specific nucleotide sequences are not listed for the sense and antisense primers since many different nucleotide sequences having the above structures can be designed.

PCR is performed using standard protocols (Ausubel, F. M. et al.: Current protocols in molecular biology, Vol. 1 (1995) John Wiley & Sons, Inc.; Sambrook, J. Fritsch, E. F. & Maniatis, T.: Molecular Cloning: A laboratory manual, 2^{nd} ed. (1989) Cold Spring Harbor Laboratory Press) and a plasmid containing the GFP1 gene as the template. After amplification, the amplified product is usually purified using agarose gel electrophoresis and kits such as Qiaquick form Qiagen, before being subcloned into an expression vector or a shuttle vector (intermediate vector), both containing the Rluc gene.

D) Counterpart Configurations:

Two further configurations that can be prepared are a modulator-BDP-FAM or a BDP-FAM-modulator, generated using similar molecular biology techniques as described for the modulator-FAM-BDP or the FAM-BDP-modulator above.

### Example VI

### Calculating Energy Transfer Efficiencies—Quantification of BDP and FAM Emissions

A 2-detector TopCount was modified (BRETCount) in order to measure BRET. Bandpass interference filters were positioned under each TopCount PMT (between the PMT and the detector mounting plate). Furthermore, the TopCount software was modified to allow each well of a microtiterplate to be read by each PMT. These modifications allow the sequential detection of light emitted at specific wavelengths from the samples. For each well (or sample) two light output values were obtained that corresponded to each optical filter. TopCount is a temperature controlled instrument which is particularly useful when performing cell-based assays or time-course experiments.

For the examples presented herein, the bandpass interference filters were designed based on emission spectra of BDP and FAM, as indicated below:

1. for the acceptor emission (fluorophore): 410DF80 (Omega Optical Inc, Brattleboro Vt.)

2. for the donor emission (bioluminescent protein): 515/50 nm (Chroma Technology Corp., Brattleboro Vt.)

Alternative optical filter combinations can be designed, depending on the BDP and FAM used in the assay.

In this example, BRET is measured by dividing the light output for the 515 nm filters by the 410 mn filters (acceptor/donor). The instrument parameters used for the BRETCount were:

Assay temperature: 25° C.

Single Photon Counting mode

Read length of 2 sec for each PMT

Cells (CHO-K1) were transfected using LipofectAMINE (Life Technologies, Rockville Md.) with either pRL-CMV DNA (that constitutively expresses Rluc) or a vector pCDNA3.1/Rluc::GFPuv) constitutively expressing the fusion construct Rluc::GFPuv (GFPuv comes from Clontech, Palo Alto Calif.). Two days post-transfection, cells were harvested, counted and distributed into 96-well plates (white Optiplate from Packard Instruments, Meriden Conn.) at a density of 50 000 cells per well in PBS+Ca²⁺+Me²⁺+glucose+2 μg/ml aprotinin. Typically transfection efficiencies were in the range of 50-60% (calculated by counting GFP fluorescent cells under a fluorescence microscope) when performed according to the manufacturer's protocol. Coelenterazine 400a was then added (to a final concentration of 5 μM) to each well to initiate the bioluminescence reaction. Light outputs were determined using the BRETCount with the 400 nm optical filter (donor emission) and the 510 nm optical filter (acceptor emission) and using the normal TopCount (ie. without the use of any filter), which allows the determination of light output for all wavelengths (open spectrum). Eight measurements were made for each type of transfected cells and for each type of measurement for a total of 48 measurements per assay. Table 6 shows the mean values for the different assay conditions. Instrument parameters for BRETCount were 25° C., with a read time of 2 sec in photon counting mode; and for the TopCount, 19° C., with a read time of 1 sec in photon counting mode.

The invention being thus described, it will be obvious that the same may be varied in many ways. Such variations are not to be regarded as a departure from the spirit and scope of the invention, and all such modifications as would be obvious to one skilled in the art are intended to be included within the scope of the claims.

TABLE 1
Exemplary BRET BDP and FAM Combinations

| | | Substrate | | Wavelength |
|---|---|---|---|---|
| | | wavelength | | of acceptor |
| BDP | Substrate | (peak) | FAM | (Ex/Em) |
| Rluc | Coelenterazine | 470 nm | Fluorescein | 490/525 nm |
| | Wild type | | | |
| Rluc | Coelenterazine | 470 nm | Acridine | 470/550 nm |
| | Wild type | | yellow | |
| Rluc | Coelenterazine | 470 nm | Nile red | 485/525 nm |
| | Wild type | | | |
| Rluc | Coelenterazine | 442 nm | Lucifer yellow | 428/540 nm |
| | cp | | | |
| Rluc | Coelenterazine | 400 nm | Quin-2 | 365/490 nm |
| | 400 | | | |
| Rluc | Coelenterazine | 400 nm | Dansylchloride | 380/475 nm |
| | 400 | | | |
| Firefly | luciferin | 560 nm | Cyanine Cy3 | 575/605 |
| luciferase | | | | |
| Firefly | luciferin | 560 nm | Texas red | 590/615 |
| luciferase | | | | |

TABLE 2

| | Final quantity or final |
|---|---|
| Parameters | concentration |
| DNA template | 10 ng |
| PFU buffer 10X (from Stratagene) | 1x |
| dNTPs 10 mM | 250 μM |
| Primer sense (1 mM) | 500 μM |
| Primer antisense (1 mM) | 500 μM |
| Milli-Q grade water | to complete to 50 μl |
| PFU enzyme* | 2.5 U |

*added last during the hot start procedure

TABLE 3

| | Intensity* | Intensity* | Intensity* |
|---|---|---|---|
| | (CPS) at | (CPS) at | (CPS) open |
| Constructs | 400 nm | 510 nm | spectrum |
| Rluc (pRL-CMV) | 1933 | 738 | 9153 |
| Rluc::GFPuv | 1008 | 944 | 15770 |
| (pcDNA3.1/Rluc::GFPuv) | | | |

TABLE 4

| | Ratio | Ratio | Ratio | Ratio |
|---|---|---|---|---|
| Constructs | eq. 1 | eq. 2 | eq. 3 | eq. 4 |
| Rluc (pRL-CMV) | 0.382 | 2.619 | 0.0877 | 3.734 |
| Rluc::GFPuv | 0.936 | 1.068 | 0.0637 | 14.637 |
| (pcDNA3.1/Rluc::GFPuv) | | | | |

We claim:

1. A bioluminescence resonance energy transfer (BRET) system comprising a fusion protein comprising:
(a) a bioluminescent donor protein (BDP);
(b) a fluorescent acceptor molecule (FAM) that can accept the energy from the BDP when they are associated, in the presence of the appropriate substrate; and
(c) a modulator,
wherein said BDP, FAM and modulator are fused such that a physical change in the modulator influences the energy transfer efficiency between the BDP and the FAM.

2. The BRET system according to claim 1, wherein said physical change in the modulator is selected from the list consisting of: cleavage, chemical modification, enzymatic modification, conformational change, binding of one or more molecule(s), binding of one or more analyte(s);

3. The BRET system according to claim 1, wherein the modulator is fused either genetically or chemically to the BDP and FAM.

4. The BRET system according to claim 1, wherein the modulator is chemically attached to a linker molecule that links the FAM to the BDP.

5. The BRET system according to claim 1, wherein the modulator is attached to the FAM which is attached to the BDP.

6. The BRET system according to claim 1, wherein the modulator is attached to the BDP which is attached to the FAM.

7. The BRET system according to claim 1, wherein the modulator is genetically inserted at the amino or carboxy terminus of the FAM which is attached to the BDP.

8. The BRET system according to claim 1, wherein the modulator is genetically inserted at the amino or carboxy terminus of the BDP which is attached to the FAM.

9. The BRET system according to claim 1, wherein the modulator is an enzyme substrate.

10. The BRET system according to claim 1, wherein the modulator is a selected from the list consisting of: a protease recognition site, a protease cleavage site, a DNA restriction enzyme recognition site, a DNA restriction enzyme cleavage site, a phosphorylation site, a glycosylation site, an ion binding domain, a second messenger binding site, an enzyme substrate site, a methylation site,a lipid binding site, a sulfation site, an isoprenylation site, an allosteric site, or any post translational modification site or a fragment thereof of any of these sites.

11. The BRET system according to claim 1, wherein the BDP is an enzyme that will act on the substrate to generate a luminescent molecule.

12. The BRET system according to claim 11, wherein the BDP has luciferase activity.

13. The BRET system according to claim 11, wherein the BDP is Renilla luciferase, Firefly luciferase, Gaussia luciferase, Aequorin, or any enzyme having bioluminescent activity.

14. The BRET system according to claim 11, wherein the enzyme is β-galactosidase, horseradish peroxidase, alkaline phophatase, β-glucuronidase or β-glucosidase.

15. The BRET system according to claim 1, wherein the FAM is green fluorescent protein, fluorescein, acridine yellow, nile red, lucifer yellow, quin-2, dansyl chloride, cyanine Cy3 or Texas red.

16. The BRET system according to claim 1, wherein the FAM is the green fluorescen protein or a mutant thereof, or the red fluorescent protein or mutant thereof.

17. The BRET system according to claim 1, additionally comprising a substrate which when acted upon by the BDP will generate luminescence.

18. The BRET system according to claim 17, wherein the substrate is luciferin, coelenterazine, a derivative of coelenterazine or related compounds.

19. Use of the BRET system of claim 1 as an apoptotic sensor, wherein said modulator is a caspase cleavage site.

20. Use of the BRET system of claim 1 to detect kinase activity, wherein said modulator is a phosphorylation site.

21. Use of the BRET system of claim 1 to detect energy transfer in cell free system.

22. Use of the BRET system of claim 1 in a host cell by introducing the fusion molecule into live cells.

23. The use according to claim 22, wherein said introduction is by microinjection or molecular carrier technology.

24. A recombinant nucleic acid encoding a fusion protein comprising a bioluminescent donor protein (BDP), a fluorescent acceptor molecule (FAM) that can accept the energy from the BDP when they are associated in the presence of the appropriate substrate, and a modulator, wherein said BDP, FAM and modulator are fused such that a physical change in the modulator influences the energy transfer between the BDP and the FAM.

25. A vector comprising the recombinant nucleic acid according to claim 21.

26. A host cell comprising the recombinant nucleic acid according to claim 21 or the vector of claim 22.

27. A method of producing a bioluminescence resonance energy transfer (BRET) system comprising:
(a) genetically engineering a fusion gene encoding a bioluminescent donor protein (BDP) fused with a fluorescent acceptor molecule (FAM), that can accept the energy from the BDP when they are associated in the presence of the appropriate substrate, and a modulator; and
(b) expressing said fusion gene to produce a fusion protein,
wherein said BDP, FAM and modulator are fused such that activity upon the modulator influences the energy transfer efficiency between of the BDP and the FAM.

28. A method of producing a bioluminescence resonance energy transfer (BRET) system comprising:
chemically linking a bioluminescent donor protein (BDP) to a fluorescent acceptor molecule (FAM), that can accept the energy from the BDP when they are associated in the presence of the appropriate substrate, and a modulator wherein said BDP, FAM and modulator are linked such that a physical change in the modulator influences energy transfer efficiency between the BDP and the FAM.

29. A method of producing a bioluminescence resonance energy transfer (BRET) system comprising:
(a) producing a fusion protein from the recombinant nucleic acid of claim 21, the vector of claim 22 or the host cell of claim 23; and
(b) purifying said fusion protein and optionally
(c) chemically modifying the purified fusion protein.
